(19) United States
(12) Patent Application Publication
(10) Pub. No.: US 2026/0070960 A1
(43) Pub. Date: Mar. 12, 2026

(54) SINGLE DOMAIN ANTIBODIES TARGETING HPV E6/E7 ONCOGENIC PEPTIDE/MHC COMPLEXES

(71) Applicant: The U.S.A., as represented by the Secretary, Department of Health and Human Services, Bethesda, MD (US)

(72) Inventors: Mitchell Ho, Urbana, MD (US); Zhijian Duan, Columbia, MD (US); Christian S. Hinrichs, New Brunswick, NJ (US)

(73) Assignee: The U.S.A., as represented by the Secretary, Department of Health and Human Services, Bethesda, MD (US)

(21) Appl. No.: 19/107,498

(22) PCT Filed: Aug. 30, 2023
(86) PCT No.: PCT/US2023/073144

§ 371 Date: Feb. 27, 2025

Related U.S. Application Data
(60) Provisional application No. 63/374,307, filed on Sep. 1, 2022.

(51) Int. Cl. C07K 16/08; A61K 40/11; A61K 40/31; A61K 40/46; A61K 47/68; A61K 47/69; A61P 35/00; C07K 14/705; C07K 14/725; C12N 5/0783; G01N 33/569

(52) U.S. Cl. CPC C07K 16/084; A61K 40/11; A61K 40/31; A61K 40/46; A61K 47/6803; A61K 47/6839; A61K 47/6929; A61P 35/00; C07K 14/7051; C07K 14/70517; C07K 14/70578; C12N 5/0636; G01N 33/56983; C07K 2317/22; C07K 2317/53; C07K 2317/565; C07K 2317/569; C07K 2317/73; C07K 2317/92; C07K 2319/02; C07K 2319/03; G01N 2333/025; G01N 2469/10

(57) ABSTRACT

Single-domain antibodies that specifically bind human papillomavirus (HPV) E6 or E7 oncogenic peptides in complex with human major histocompatibility complex (MHC) proteins are described. The E6-MHC-specific and E7-MHC-specific single-domain antibodies were isolated from dromedary camel (V_{H}H) antibody libraries by panning the library with an E6- or E7-derived peptide in complex with HLA-A*02:01. Use of the single-domain antibodies for the detection and treatment of HPV-associated cancers and pre-cancerous lesions is also described.

### CROSS REFERENCE TO RELATED APPLICATIONS

This application claims the benefit of U.S. Provisional Application No. 63/374,307, filed Sep. 1, 2022, which is herein incorporated by reference in its entirety.

### ACKNOWLEDGMENT OF GOVERNMENT SUPPORT

This invention was made with government support under project numbers Z01 BC010891 and ZIA BC011943 awarded by the National Institutes of Health. The government has certain rights in the invention.

### FIELD

This disclosure concerns single-domain monoclonal antibodies that specifically bind human papillomavirus (HPV) E6 or HPV E7 oncogenic peptides in complex with major histocompatibility complex (MHC) proteins, and use of the single-domain antibodies as anti-cancer agents.

### INCORPORATION OF ELECTRONIC SEQUENCE LISTING

The electronic sequence listing, submitted herewith as an XML file named 4239-108686-02.xml (21,494 bytes), created on Aug. 1, 2023, is herein incorporated by reference in its entirety.

### BACKGROUND

Current antibody-based cancer immunotherapies target a limited number of tumor specific surface proteins. However, most oncogenic drivers are intracellular proteins that are not easily accessed by antibody-based immunotherapy (Duan and Ho, Mol Cancer Ther 20(9):1533-1541, 2021; Trenevska et al., Front Immunol 8:1001, 2017). The only portions of these oncogenic proteins that are accessible to the immune system are peptide fragments presented by the major histocompatibility complex (MHC; also called human leucocyte antigen (HLA)) on the cell surface. The peptides originate from various tumor antigens, including intracellular tumor antigens such as viral oncogene products, transcription factors, oncofetal proteins, cancer-testis antigens, and neoantigens from mutated oncogenes (Duan and Ho, Mol Cancer Ther 20(9):1533-1541, 2021; Trenevska et al., Front Immunol 8:1001, 2017; Leko and Rosenberg, Cancer Cell 38(4): 454-472, 2020; Pearlman et al., Nat Cancer 2021; 2(5):487-497, 2021). The peptide-MHC (pMHC) complexes can be targeted by engineered T cell receptor (TCR) therapy. However, the TCR may be highly individualized and limited to MHC class (Zhao and Cao, Front Immunol 2019; 10:2250, 2019; Qin et al., Cells 10(4):808, 2021). One alternative is TCR-like or TCR mimic (TCRm) antibodies that recognize pMHC complexes and mimic the binding of TCR to the complexes. TCRm antibodies expand the range of therapeutic targets to both extracellular and intracellular proteins and thus have broad clinical potential.

Human papillomavirus (HPV) antigens E6 and E7 are oncogenic and constitutively expressed by tumors, but not by healthy tissues (Draper et al., Clin Cancer Res 21(19):4431-4439, 2015; Jin et al., JCI Insight 3(8):e99488, 2018; Pal et al., Front Microbiol 10:3116, 2019). HPV has been linked to cancers of the uterine cervix, oropharynx, anus, vulva, vagina, and penis. Although HPV vaccines aid in the prevention of HPV-associated cancers, there are still more than 5,000 deaths caused by HPV-associated cancers each year in the US and cervical cancer continues to be the second leading cause of cancer death in women aged 20 to 39 years (Siegel et al., CA Cancer J Clin 71(1):7-33, 2021). It is difficult to treat those malignancies, especially metastatic disease which is generally considered incurable. Although TCR gene therapy has been effective in some patients (Nagarsheth et al., Nat Med 27(3):419-425, 2021), there remains an urgent need for innovative treatments for HPV-associated cancer.

### SUMMARY

The present disclosure describes polypeptides, such as single-domain antibodies, that bind HPV E6 or HPV E7 oncogenic peptides in complex with MHC proteins. The disclosed antibodies were isolated from dromedary camel (V_{H}H) antibody libraries by panning the library with an E6- or E7-derived peptide in complex with HLA-A*02:01. The disclosed polypeptides can be used, for example, in the development of therapeutics to treat HPV-associated cancers or pre-cancerous lesions.

Provided herein are polypeptides (for example, single-domain monoclonal antibodies) that bind, such as specifically bind, HPV E6 peptide in complex with MHC (“E6-MHC”) or HPV E7 peptide in complex with MHC (“E7-MHC”). In some aspects, the polypeptide that binds E6-MHC includes the complementarity determining region (CDR) sequences of camel nanobody F5 or G9, or the polypeptide that binds E7-MHC includes the CDR sequences of camel nanobody H3, H7 or H9.

Also provided herein are conjugates that include a disclosed polypeptide (or the CDR sequences of a disclosed polypeptide). In some aspects, provided are fusion proteins (such as Fc fusion proteins), chimeric antigen receptors (CARs), CAR-expressing immune cells (such as T cells, B cells, natural killer cells, macrophages and induced pluripotent stem cells (iPSCs)), soluble T cell receptors (TCRs), immunoconjugates (such as immunotoxins), multi-specific antibodies (such as bispecific or trispecific antibodies), antibody-drug conjugates (ADCs), antibody-nanoparticle conjugates, and antibody-photon absorber conjugates (such as IR700 conjugates for immunoPET imaging) that include a polypeptide (for example, single-domain monoclonal antibody) disclosed herein.

Further provided are compositions that include one or more (such as at least two or at least three) different E6-MHC- or E7-MHC-specific polypeptides disclosed herein. In some examples, such a composition includes a pharmaceutically acceptable carrier. In some examples, such a composition is lyophilized.

Also provided herein are nucleic acid molecules and vectors encoding the disclosed polypeptides (for example, single-domain antibodies), fusion proteins (such as Fc fusions or nanobodies converted to IgG, IgM or IgA), CARs, soluble TCRs, immunoconjugates (such as immunotoxins), and multi-specific antibodies (such as bi-specific antibodies) disclosed herein. Isolated cells that include a nucleic acid or vector encoding an E6-MHC-specific or E7-MHC-specific polypeptide or conjugate thereof are further provided.

Compositions and kits that include a pharmaceutically acceptable carrier and an E6-MHC-specific or E7-MHC-specific polypeptide, fusion protein, CAR, soluble TCR, immunoconjugate, ADC, multi-specific antibody, antibody-nanoparticle conjugate, isolated nucleic acid molecule or vector disclosed herein are also provided by the present disclosure. Also provided are solid supports, such as beads (e.g., glass, magnetic, plastic), multiwell plates, paper, or nitrocellulose that include one or more E6-MHC-specific or E7-MHC-specific polypeptides (such as single-domain monoclonal antibodies) provided herein. In some aspects, the composition includes recombinant yeast or bacteria that express a disclosed antibody.

Also provided is a method of treating an HPV-associated cancer or pre-cancerous lesion in a subject. In some aspects, the method includes administering to the subject a therapeutically effective amount of a polypeptide (for example, a single-domain monoclonal antibody) disclosed herein, or administering to the subject a therapeutically effective amount of a fusion protein, CAR (or CAR T cells, CAR B cells, CAR NK cells, CAR macrophages or CAR iPSCs), soluble TCR, immunoconjugate (such as an immunotoxin), ADC, multi-specific antibody, or antibody-nanoparticle conjugate that includes a polypeptide disclosed herein, or a nucleic acid molecule or vector encoding a disclosed polypeptide. In some examples, the HPV is HPV16 or HPV18. Such treatments can be combined with other therapeutic agents, such as other anti-cancer therapies and/or an anti-viral therapy.

Also provided are solid supports that include a one or more of the polypeptides disclosed herein. In some aspects, the solid support includes a bead, multiwell plate, or nitrocellulose. Use of the solid support for detecting HPV-infected cells in a sample is also provided.

Methods of detecting HPV-infected cells in a sample, and methods of diagnosing a subject as having an HPV infection, are further provided. In some aspects, the methods include contacting a sample containing cells from the subject with a polypeptide (for example, a single-domain monoclonal antibody) disclosed herein, and detecting binding of the polypeptide to cells in the sample. In specific examples, the HPV is HPV16 or HPV18.

The foregoing and other features of this disclosure will become more apparent from the following detailed description of several aspects which proceeds with reference to the accompanying figures

### BRIEF DESCRIPTION OF THE DRAWINGS

FIGS. 1A-1H: TCRm nanobodies F5 and G9 were identified by phage panning from dromedary camel single domain antibody libraries. (FIG. 1A) Schematic of monomer synthesis. (FIG. 1B) Schematic depiction of the phage panning process, nanobody discovery and application of the chimeric antigen receptor (CAR) format. (FIG. 1C) Table listing input and output phage numbers in the four rounds of phage panning. (FIG. 1D) Polyclonal phage ELISA results from phage panning. (FIG. 1E) Monoclonal phage ELISA of binders F5 and G9 against pMHC-E6, pMHC-E7 and control antigens. (FIG. 1F) SDS-PAGE gel image of the two binders F5 and G9 from protein purification. (FIG. 1G) ELISA data showing binding of F5 and G9 to pMHC-E6, pMHC-E7 and control antigens. (FIG. 1H) KDs of F5 and G9 binding to pMHC-E6 complex by bio-layer interferometry (BLI) technology. *p<0.05.

FIGS. 2A-2D: F5 and G9 nanobodies exhibit different binding properties for the complexes expressed on cells. (FIGS. 2A and 2B) Peptide pulsing on human T2 lymphoblastic cells. E6, E7 and A2 (influenza virus) peptides (50 μM) were pulsed on 1 million T2 cells overnight and the cells were stained with HLA-A*02 antibody for FACS (FIG. 2A). Cells were also stained with the nanobodies to measure binding by FACS (FIG. 2B). (FIG. 2C) Binding of F5 and G9 to the complexes on 293, 293E6 and 293E7 cells by FACS. (FIG. 2D) Binding of F5 and G9 nanobodies on tumor cell lines Caski and SCC90.

FIGS. 3A-3D: The C-terminal residues of E6 peptide were involved in the binding of F5 to the complex. (FIGS. 3A-3C) T2 cells were pulsed with 50 μM E6 peptide (SEQ ID NO: 11) or one of 10 mutated peptides (SEQ ID NOs: 12-21), each of which have a different single mutation to alanine. The cells were then examined by FACS for the expression of the complex and F5 binding at 5 μg/ml and 20 μg/ml. (FIG. 3D) Molecular models showing docking of F5 to the pMHC-E6 by i-TASSER and ClusPro.

FIGS. 4A-4E: F5 based CAR-T cells showed specific and efficient cytotoxicity against target cells in vitro. (FIG. 4A) Schematic design of CAR construction. The CAR includes the F5 or G9 V_{H}H, a CD8α hinge region, a CD8α transmembrane domain, a 41BB co-stimulatory domain and a CD3ζ signaling domain. The CAR is co-expressed with a truncated form of human EGFR (hEGFRt). (FIG. 4B) Transduction efficiency of F5 and G9 CAR-T cells. (FIG. 4C) F5 or G9 CAR-T cells were cocultured with target cells 293, 293E6 or 293E7 at different effector to target (E/T) ratios (3:1, 6:1, 12.5:1 and 25:1) for 24 hours. Cell viability was measured by luciferase activity. (FIG. 4D) F5 CAR-T cells were cocultured with Caski target cells at different E/T ratios (3:1, 6:1, 12.5:1 and 25:1) for 24 or 48 hours. Cell viability was measured by luciferase activity. (FIG. 4E) The supernatant from Caski and CAR-T coculture at E/T ratio 12.5:1 was collected and analyzed by multiplex cytokine assays using FACS. *p<0.05.

FIGS. 5A-5H: F5 CAR-T cells inhibited tumor growth in a Caski xenograft model. (FIG. 5A) Schematic timeline of tumor inoculation, CAR-T injection and tumor monitoring. (FIG. 5B) Tumor growth curve following infusion of F5 CAR-T cells, antigen mismatched CD19 CAR-T cells, or mock cells measured by caliper for seven weeks following infusion. (FIG. 5C) Tumor images taken at 42 days after CAR-T infusion. (FIG. 5D) Body weight measured weekly at days 14, 21, 28, 35 and 42 following CAR-T infusion. (FIG. 5E) The spleens of treated mice were harvested and cultured with IL-7, IL-15 and IL-21 for expansion of CAR-T cells. The cells were stained for truncated human EGFR and CD3 by FACS. (FIG. 5F) The expanded CAR-T cells were cocultured with Caski cells at different E/T ratios for 24 hours and the cell viability was measured by luciferase activity. (FIG. 5G) The supernatant from Caski and CAR-T cocultured at E/T ratio 12.5:1 was collected and analyzed by multiplex cytokine assays using FACS. (FIG. 5H) Cells were stained with anti-PD-1 antibody and evaluated by FACS. *p<0.05.

FIGS. 6A-6C: The C-terminal residues of E6 peptide are involved in the binding of G9 to the complex. T2 cells were pulsed with 50 μM E6 peptide (SEQ ID NO: 11) or one of 10 mutated E6 peptides (SEQ ID NOs: 12-21), which each have a single mutation to alanine at a different position. Cells were then examined by FACS for the expression of the complex and G9 binding at 5 μg/ml and 20 μg/ml.

FIGS. 7A-7G: F5 CAR-T cells inhibited tumor growth in an SS4050 xenograft mouse model. (FIG. 7A) Schematic of the study design, including tumor inoculation at Day 0, CAR-T cell infusion on Day 7, and tumor monitoring for three weeks post-infusion. (FIG. 7B) Tumor volume in mock-treated, F5 CAR-T cell-treated and CD19 CAR-T cell-treated mice at Days 1, 7, 14 and 21 post CAR-T cell infusion. (FIG. 7C) Tumor images after final timepoint. (FIG. 7D) Flow cytometry plots measuring expression of epidermal growth factor receptor (EGFR) and CD3 by spleen cells of F5 CAR-T cell-treated mice. Spleens of treated mice were harvested and cultured in media containing IL-7, IL-15 and IL-21 for expansion of CAR-T cells and stained for EGFR and CD3. (FIG. 7E) The expanded CAR-T cells were cocultured with Caski cells at different E/T ratio for 24 hours and the cell viability was measured by luciferase activity. (FIG. 7F) Flow cytometry plots showing PD-L1 expression in SS4050 and Caski cells. (FIG. 7G) The isolated spleen cells were stained with anti-PD-1 antibody by FACS. *p<0.05.

### SEQUENCE LISTING

The nucleic acid and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing: SEQ ID NO: 1 is the amino acid sequence of single-domain antibody F5.
SEQ ID NO: 2 is the amino acid sequence of single-domain antibody G9.
SEQ ID NO: 3 is the amino acid sequence of single-domain antibody H3.
SEQ ID NO: 4 is the amino acid sequence of single-domain antibody H7.
SEQ ID NO: 5 is the amino acid sequence of single-domain antibody H9.
SEQ ID NO: 6 is a nucleic acid sequence encoding single-domain antibody F5.
SEQ ID NO: 7 is a nucleic acid sequence encoding single-domain antibody G9.
SEQ ID NO: 8 is a nucleic acid sequence encoding single-domain antibody H3.
SEQ ID NO: 9 is a nucleic acid sequence encoding single-domain antibody H7.
SEQ ID NO: 10 is a nucleic acid sequence encoding single-domain antibody H9.
SEQ ID NO: 11 is the amino acid sequence of an E6 peptide.
SEQ ID NOs: 12-21 are amino acid sequences of mutant E6 peptides.
SEQ ID NO: 22 is the amino acid sequence of an E7 peptide.

### DETAILED DESCRIPTION

The present disclosure describes TCRm single-domain antibodies that target an epitope of the oncogenic viral antigens HPV E6 or HPV E7 in the context of HLA-A*02:01. HPV viral antigens E6 and E7 are oncogenic and constitutively expressed by tumors, but not expressed by healthy tissue, making them targets for TCRm antibody development. Although engineered T cell therapy has demonstrated efficacy and safety in patients with HPV-associated malignancies, especially with metastatic tumors, tumor resistance and immune escape can lead to ineffectiveness of the treatment in some patients (Draper et al., Clin Cancer Res 21(19):4431-4439, 2015; Jin et al., JCI Insight 3(8):e99488, 2018; Nagarsheth et al., Nat Med 27(3):419-425, 2021). In addition, widespread implementation is constrained by the need for patients' autologous cells and for sophisticated manipulation of cells in an individualized manner.

The presence of neoantigens on the tumor cell surface is the foundation for the development of TCRm antibodies. In HPV16+ related epithelial cancers, both E6-MHC and E7-MHC are present in tumor cells (Draper et al., Clin Cancer Res 21(19):4431-4439, 2015; Jin et al., JCI Insight 3(8):e99488, 2018). TCRm antibodies have been developed to target aberrantly expressed intracellular oncogenic and tumor-associated antigens (TAAs) such as Wilms tumor 1 (WT1), gp100, MAGE-A3, Melan-A, and NY-ESO-1 (Duan and Ho, Mol Cancer Ther 20(9):1533-1541, 2021; Trenevska et al., Front Immunol 8:1001, 2017; Dao et al., Sci Transl Med 5(176):176ra33, 2013; Chinnasamy et al., J Immunol 186(2):685-696, 2011). Studies have also described the development of TCRm antibodies that recognize the mutation-associated neoantigens derived from TP53 or KRAS (Douglass et al., Sci Immunol 6(57):eabd5515, 2021; Hsiue et al., Science 371(6533):eabc8697, 2021; Liu et al., Clin Cancer Res 23(2):478-488, 2017).

Single-domain antibodies are small and can be used as therapeutic and/or diagnostic agents, or as modular building blocks for multi-domain constructs, antibody-drug conjugates, immunotoxins, or chimeric antigen receptors (Muyldermans, Annu Rev Anim Biosci 9:401-421, 2021; Muyldermans, Annu Rev Biochem 82:775-797, 2013; English et al., Antib Ther 3(1):1-9, 2020). Described herein are two E6-MHC specific (F5 and G9) and three E7-MHC specific (H3, H7 and H9) camel V_{H}H antibodies isolated from dromedary camel V_{H}H libraries. The construction of phage-displayed dromedary camel (Camelus dromedarius) V_{H}H single domain phage libraries was described previously (Hong et al., Proc Natl Acad Sci USA 119(18):e2201433119, 2022). T cells expressing a chimeric antigen receptor (CAR) that includes F5 as the antigen-binding domain, killed HPV16+ cervical tumor cells in vitro and in vivo through the release of key cytolytic cytokines. These results demonstrate that nanobodies that specifically bind E6-MHC or E7-MHC complexes can be used in the development of therapeutics for the treatment of HPV-associated cancers and pre-cancerous lesions.

### I. Abbreviations

ADC antibody-drug conjugate
CAR chimeric antigen receptor
CDR complementarity determining region
FACS fluorescence activated cells sorting
FR framework
HPV human papillomavirus
MHC major histocompatibility complex
NK natural killer
PE Pseudomonas exotoxin
PET positron emission tomography
pMHC peptide MHC
TCR T cell receptor
TCRm TCR mimic

### II. Terms and Methods

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a.” “an,” and “the,” refer to both the singular as well as plural, unless the context clearly indicates otherwise. For example, the term “an antibody” includes singular or plural antibodies and can be considered equivalent to the phrase “at least one antibody.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: To provide or give a subject an agent, such as a polypeptide (e.g., a single domain monoclonal antibody) provided herein, by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Antibody: A polypeptide ligand comprising at least one variable region that recognizes and binds (such as specifically recognizes and specifically binds) an epitope of an antigen, such as an HPV peptide (e.g., an E6 or E7 peptide) in complex with an MHC molecule. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE. Antibody isotypes not found in mammals include IgX, IgY, IgW and IgNAR. IgY is the primary antibody produced by birds and reptiles and is functionally similar to mammalian IgG and IgE. IgW and IgNAR antibodies are produced by cartilaginous fish, while IgX antibodies are found in amphibians.

Antibody variable regions contain framework regions (FR) and hypervariable (HV) regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and A1-Lazikani et al., JMB 273,927-948, 1997; the “Chothia” numbering scheme), Kunik et al. (see Kunik et al., PLoS Comput Biol 8: e1002388, 2012; and Kunik et al., Nucleic Acids Res 40 (Web Server issue): W521-524, 2012; “Paratome CDRs”) and the ImMunoGene Tics (IMGT) database (see, Lefranc, Nucleic Acids Res 29:207-9, 2001; the “IMGT” numbering scheme). The Kabat, Paratome and IMGT databases are maintained online.

A “single-domain antibody” refers to an antibody having a single domain (a variable domain) that is capable of specifically binding an antigen, or an epitope of an antigen, in the absence of an additional antibody domain. Single-domain antibodies include, for example, V_{H} domain antibodies, V_{NAR} antibodies, camelid V_{H}H antibodies, and V domain antibodies. V_{NAR} antibodies are produced by cartilaginous fish, such as nurse sharks, wobbegong sharks, spiny dogfish and bamboo sharks. Shark V_{NAR} are comprised of the following regions (N-terminal to C-terminal): FR1-CDR1-FR2-HV2-FR3a-HV4-FR3b-CDR3-FR4. The positions of CDR1 and CDR3 of V_{NAR} antibodies can be determined, for example, using IMGT. HV2 and HV4 can be determined, for example, using annotation described in Stanfield et al. (Science 305:1770-1773, 2004) and Fennell et al. (J Mol Biol 400:155-170, 2010). Camelid V_{H}H antibodies are produced by several species including camel, llama, alpaca, dromedary, and guanaco, which produce heavy chain antibodies that are naturally devoid of light chains. Camel V_{H}H are comprised of the following regions (N-terminal to C-terminal): FR1-CDR1-FR2-CDR2-FR3-CDR3-FR4. Camel V_{H}H CDR residues can be determined, for example, according to IMGT, Kabat or Paratome.

A “monoclonal antibody” is an antibody produced by a single clone of lymphocytes or by a cell into which the coding sequence of a single antibody has been transfected. Monoclonal antibodies include humanized monoclonal antibodies.

A “chimeric antibody” has framework residues from one species, such as human, and CDRs (which generally confer antigen binding) from another species.

A “humanized” antibody is an immunoglobulin including a human framework region and one or more CDRs from a non-human (for example a camel, llama, mouse, rabbit, rat, shark or synthetic) immunoglobulin. The non-human immunoglobulin providing the CDRs is termed a “donor,” and the human immunoglobulin providing the framework is termed an “acceptor.” In one aspect, all CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A humanized antibody binds to the same antigen as the donor antibody that provides the CDRs. Humanized or other monoclonal antibodies can have additional conservative amino acid substitutions which have substantially no effect on antigen binding or other immunoglobulin functions.

Antibody-drug conjugate (ADC): A molecule that includes an antibody (or antigen-binding fragment of an antibody) conjugated to a drug, such as an anti-viral agent or a cytotoxic agent. ADCs can be used to specifically target a drug to particular cells through specific binding of the antibody to a target antigen expressed on the cell surface. Exemplary drugs for use with ADCs include anti-viral agents (such as cidofovir, biphenylsulphonacetic acid, histone deacetylase (HDAC) inhibitors, or derivatives of nordihydroguaiaretic acid (NDGA; such as tetra-O-methyl NDGA or tetra-acetyl NDGA)), anti-microtubule agents (such as maytansinoids, auristatin E and auristatin F) and interstrand crosslinking agents (for example, pyrrolobenzodiazepines; PBDs). In some cases, the ADC is a bi-specific ADC, which is comprised of two monoclonal antibodies or antigen-fragments thereof, each directed to a different antigen or epitope (e.g., to E6-MHC and E7-MHC complexes), conjugated to a drug. In one example, the agent attached to the antibody is IRDye® 700 DX (IR700, Li-cor, Lincoln, NE), which can then be used with near infrared light NIR light to kill target cells to which the antibody binds (photoimmunotherapy; see for example U.S. Pat. Nos. 8,524,239 and 10,538,590). For example, amino-reactive IR700 can be covalently conjugated to an antibody using the NHS ester of IR700.

Binding affinity: Affinity of an antibody for an antigen. In one aspect, affinity is calculated by a modification of the Scatchard method described by Frankel et al., Mol. Immunol., 16:101-106, 1979. In another aspect, binding affinity is measured by an antigen/antibody dissociation rate. In another aspect, a high binding affinity is measured by a competition radioimmunoassay. In another aspect, binding affinity is measured by ELISA. In some aspects, binding affinity is measured using the Octet system (Creative Biolabs), which is based on bio-layer interferometry (BLI) technology. In other aspects, Kd is measured using surface plasmon resonance assays using a BIACORES-2000 or a BIACORES-3000 (BIAcore, Inc., Piscataway, N.J.). In other aspects, antibody affinity is measured by flow cytometry. An antibody that “specifically binds” an antigen (such as E6-MHC complexes) is an antibody that binds the antigen with high affinity and does not significantly bind other unrelated antigens.

In some examples, a single-domain monoclonal antibody (such as an anti-E6-MHC or anti-E7-MHC single-domain antibody provided herein) specifically binds to its target with a binding constant that is at least 10³ M⁻¹ greater, 10⁴ M⁻¹ greater or 10⁵ M⁻¹ greater than a binding constant for other molecules in a sample or subject. In some examples, an antibody (e.g., single-domain monoclonal antibody) has an equilibrium constant (Kd) of 10 nM or less, such as 9 nM or less, 8 nM or less, 7 nM or less, 6 nM or less, 5.7 nM or less, 5.5 nM or less, 5.3 nM or less, 5 nM or less, 4.3 nM or less, 4 nM or less, 3 nM or less, 2 nM or less, 1.5 nM or less, 1.5 nM or less, 1.4 nM or less, 1.3 nM or less, or 1.2 nM or less. For example, a monoclonal antibody binds to a target, such as the E6-MHC or E7-MHC complex, with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M, at least about 2.5×10⁻⁸, at least about 3.0×10⁻⁸, at least about 3.5×10⁻⁸, at least about 4.0×10⁻⁸, at least about 4.5×10⁻⁸, at least about 5.0×10⁻⁸ M, at least about 1×10⁻⁹ M, at least about 1.3×10⁻⁹ M, at least about 1.5×10⁻⁹ M, at least about 2×10⁻⁹ M, at least about 3×10⁻⁹ M, at least about 4×10⁻⁹ M, at least about 4.3×10⁻⁹ M, at least about 5×10⁻⁹ M, at least about 6×10⁻⁹ M, at least about 6.3×10⁻⁹ M, at least about 6.9×10⁻⁹ M, at least about 7×10⁻⁹ M, at least about 8×10⁻⁹ M, at least about 8.1×10⁻⁹ M, or at least about 10×10⁻⁹ M. In certain aspects, a specific binding agent that binds to its target has a dissociation constant (Kd) of ≤1 μM, ≤100 nM, ≤10 nM, ≤9 nM, ≤8 nM, ≤7 nM, ≤6.9 nM, ≤6.5 nM, ≤6.3 nM, ≤5 nM, ≤4 nM, ≤4.5 nM, ≤3 nM, ≤2 nM, ≤1.5 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

Bispecific antibody: A recombinant protein that includes antigen-binding fragments of two different monoclonal antibodies and is thereby capable of binding two different antigens, such as an E6-MHC complex and an E7-MHC complex. Similarly, a multi-specific antibody is a recombinant protein that includes antigen-binding fragments of at least two different monoclonal antibodies, such as two, three or four different monoclonal antibodies.

Chimeric antigen receptor (CAR): A chimeric molecule that includes an antigen-binding portion (such as single-domain antibody) and a signaling domain, such as a signaling domain from a T cell receptor (for example, CD3ζ). Typically, CARs are comprised of an antigen-binding moiety, a transmembrane domain and an endodomain. The endodomain typically includes a signaling chain having an immunoreceptor tyrosine-based activation motif (ITAM), such as CD3ζ or FcεRIγ. In some instances, the endodomain further includes the intracellular portion of at least one additional co-stimulatory domain, such as CD28, 4-1BB (CD137), ICOS, OX40 (CD134), CD27, MYD88-CD40, KIR2DS2 and/or DAP10. In some examples, the CAR is multispecific (such as bispecific) or bicistronic. A multispecific CAR is a single CAR molecule comprised of at least two antigen-binding domains (such as scFvs and/or single-domain antibodies) that each bind a different antigen or a different epitope on the same antigen (see, for example, US 2018/0230225). For example, a bispecific CAR refers to a single CAR molecule having two antigen-binding domains that each bind a different antigen. A bicistronic CAR refers to two complete CAR molecules, each containing an antigen-binding moiety that binds a different antigen. In some cases, a bicistronic CAR construct expresses two complete CAR molecules that are linked by a cleavage linker. T cells or NK cells (or other immune cells, such as macrophages or B cells) expressing a bispecific or bicistronic CAR can bind cells that express both of the antigens to which the binding moieties are directed (see, for example, Qin et al., Blood 130:810, 2017; and WO/2018/213337). In some aspects, the CAR is a two-chained antibody-T cell receptor (AbTCR) as described in Xu et al. (Cell Discovery 4:62, 2018) or a synthetic T cell receptor and antigen receptor (STAR) as described by Liu et al. (Sci Transl Med 13(586):eabb5191, 2021).

Complementarity determining region (CDR): A region of hypervariable amino acid sequence that defines the binding affinity and specificity of an antibody. The light and heavy chains of a mammalian immunoglobulin each have three CDRs, designated L-CDR1, L-CDR2, L-CDR3 and H-CDR1, H-CDR2, H-CDR3, respectively. Camel (V_{H}H) single-domain antibodies, V_{H} single-domain antibodies and V_{L} single-domain antibodies contain three CDRs, referred to as CDR1, CDR2 and CDR3.

Conservative variant: A protein containing conservative amino acid substitutions that do not substantially affect or decrease the affinity of a protein, such as an antibody to an E6-MHC or an E7-MHC complex. For example, a monoclonal antibody that specifically binds to E6-MHC or E7-MHC can include at most about 1, at most about 2, at most about 5, and most about 10, or at most about 15 conservative substitutions and specifically bind the E6-MHC or E7-MHC complex. The term “conservative variant” also includes the use of a substituted amino acid in place of an unsubstituted parent amino acid, provided that antibody specifically binds the E6-MHC or E7-MHC complex. Non-conservative substitutions are those that reduce an activity or binding to the E6-MHC or E7-MHC complex.

Conservative amino acid substitution tables providing functionally similar amino acids are well known. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form.

Cytotoxic agent: Any drug or compound that kills cells.

Cytotoxicity: The toxicity of a molecule, such as an immunotoxin, to the cells intended to be targeted, as opposed to the cells of the rest of an organism. In contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Detect: To determine if a particular agent or analyte is present or absent, and in some examples further includes quantification of the agent/analyte if detected. In some examples, the agent detected is an E6-MHC or E7-MHC complex.

Diagnostic: Identifying the presence or nature of a pathologic condition, such as an HPV infection. Diagnostic methods differ in their sensitivity and specificity. The “sensitivity” of a diagnostic assay is the percentage of diseased individuals who test positive (percent of true positives). The “specificity” of a diagnostic assay is one minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (such as severity) of a pathologic condition, such as an HPV-associated cancer.

Diagnostic imaging: Coupling antibodies and their derivatives with positron emitting radionuclides for positron emission tomography (PET) is a process often referred to as immunoPET. While full length antibodies can make good immunoPET agents, their biological half-life necessitates waiting several days prior to imaging, resulting in an increase in non-target radiation doses. Smaller, single domain antibodies, such as camel or shark nanobodies, have biological half-lives amenable to same day imaging.

Drug: Any compound used to treat, ameliorate or prevent a disease or condition in a subject. In some aspects herein, the drug is an anti-viral agent, such as an anti-HPV agent, or an anti-cancer agent.

Effector molecule: The portion of a chimeric molecule that is intended to have a desired effect on a cell to which the chimeric molecule is targeted. Effector molecule is also known as an effector moiety (EM), therapeutic agent, diagnostic agent, or similar terms. Therapeutic agents (or drugs) include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as an anti-E6-MHC antibody, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are known (see, for example, U.S. Pat. No. 4,957,735; and Connor et al., Pharm Ther 28:341-365, 1985). Diagnostic agents or moieties include radioisotopes and other detectable labels. Detectable labels useful for such purposes include radioactive isotopes such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I, fluorophores, chemiluminescent agents, and enzymes.

Framework region: Amino acid sequences interposed between CDRs (and/or hypervariable regions).

Fusion protein: A protein comprising at least a portion of two different (heterologous) proteins. In some aspects, a fusion protein includes a single-domain monoclonal antibody fused to an Fc region.

Heterologous: Originating from a separate genetic source or species.

Host cell: Cells in which a vector can be propagated and its DNA expressed. The cell may be prokaryotic or eukaryotic. In some examples, the prokaryotic cell is an E. coli cell. In some examples, the eukaryotic cell is a human cell, such as a human embryonic kidney (HEK) cell. The term also includes any progeny of the subject host cell. It is understood that all progeny may not be identical to the parental cell since there may be mutations that occur during replication. However, such progeny are included when the term “host cell” is used.

Human leukocyte antigen (HLA): Proteins encoded by the MHC gene complex. HLAs from MHC Class I include HLA-A, HLA-B, and HLA-C genes and are highly variable, with up to hundreds of variant alleles at some loci. HLA loci are named with HLA, followed by the locus (e.g., A), and a number (such as 02:01) designating a specific allele at the locus (e.g., HLA-A*02:01).

Human papillomavirus (HPV): A small, non-enveloped, double-stranded DNA virus of the Papillomaviridae family. HPV infects mucosal and cutaneous epithelial cells and can cause warts, pre-cancerous lesions or cancer. Low-risk HPV types can cause genital and anal warts, while high-risk HPV can cause cancer, such as cancer of the cervix, vagina, vulva, penis, anus or throat. Most HPV-associated cancers and pre-cancerous lesions are caused by HPV16 and HPV18. However, other high-risk HPV types include HPV31, HPV33, HPV34, HPV35, HPV39, HPV45, HPV51, HPV52, HPV56, HPV58, HPV59, HPV66, HPV68 and HPV70 (Burd, Clin Microbiol Rev 16(1):1-17, 2003). HPV E6 and HPV E7 are oncogenic proteins encoded by HPV that interfere with cellular tumor suppressor proteins.

HPV-associated cancer or pre-cancerous lesion: Any type of cancer or pre-cancerous lesion that is caused by HPV, such as a high-risk HPV type (e.g., HPV16 or HPV18). In some aspects, the HPV-associated cancer is a cervical cancer, vaginal cancer, vulvar cancer, penile cancer, anal cancer or oropharyngeal cancer.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4⁺ response or a CD8⁺ response. In another aspect, the response is a B cell response, and results in the production of specific antibodies.

Immunoconjugate: A covalent linkage of an effector molecule to an antibody (such as a single-domain monoclonal antibody) or functional fragment thereof. The effector molecule can be, for example, a detectable label, a photon absorber (such as IR700), or a toxin (to form an immunotoxin, such as an immunotoxin comprising Pseudomonas exotoxin or a variant thereof). Specific, non-limiting examples of toxins include, but are not limited to, abrin, ricin, Pseudomonas exotoxin (PE, such as PE35, PE37, PE38, and PE40), diphtheria toxin (DT), botulinum toxin, or modified toxins thereof, or other toxic agents that directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as the domain Ia of PE and the B chain of DT) and replacing it with a different targeting moiety, such as an antibody. In one aspect, an antibody is joined to an effector molecule. In another aspect, an antibody joined to an effector molecule is further joined to a lipid or other molecule, such as to increase its half-life in the body. The linkage can be either by chemical or recombinant means. In one aspect, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because immunoconjugates were originally prepared from two molecules with separate functionalities, such as an antibody and an effector molecule, they are also sometimes referred to as “chimeric molecules.” The term “chimeric molecule,” as used herein, therefore refers to a targeting moiety, such as a ligand or an antibody, conjugated (coupled) to an effector molecule. The term “conjugated” or “linked” refers to making two polypeptides into one contiguous polypeptide molecule.

Immunoliposome: A liposome with antigen-binding polypeptides (such as antibodies or antibody fragments) conjugated to its surface. Immunoliposomes can carry cytotoxic agents or other drugs to antibody-targeted cells, such as virus-infected cells.

Isolated: An “isolated” biological component, such as a nucleic acid, protein (including antibodies) or organelle, has been substantially separated or purified away from other biological components in the environment (such as a cell) in which the component occurs, for example other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids. In some examples, an isolated biological component is at least 90% pure, at least 95%, at least 98%, at least 99%, at least 99.9%, at least 99.99% or 100% pure.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “labeled antibody” refers to incorporation of another molecule in the antibody. For example, the label is a detectable marker, such as the incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Various methods of labeling polypeptides and glycoproteins are known and may be used. Examples of labels for polypeptides include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y, ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some aspects, labels are attached by spacer arms of various lengths to reduce potential steric hindrance.

Linker: In some cases, a linker is a peptide within an antibody binding fragment (such as an Fv fragment) which serves to indirectly bond the variable heavy chain to the variable light chain. “Linker” can also refer to a peptide serving to link a targeting moiety, such as an antibody, to an effector molecule, such as a cytotoxin or a detectable label. The terms “conjugating,” “joining,” “bonding” or “linking” refer to making two polypeptides into one contiguous polypeptide molecule, or to covalently attaching a radionuclide or other molecule to a polypeptide, such as an antibody. The linkage can be either by chemical or recombinant means. “Chemical means” refers to a reaction between the antibody moiety and the effector molecule such that there is a covalent bond formed between the two molecules to form one molecule.

Major histocompatibility complex (MHC) Class I: MHC class I molecules are heterodimers formed from two non-covalently associated proteins, the HLA heavy chain and β2-microglobulin. The HLA heavy chain includes three distinct domains, α1, α2 and α3. The three-dimensional structure of the α1 and α2 domains form the groove into which a peptide antigen fits for presentation to T cells. The α3 domain is an immunoglobulin-fold like domain that contains a transmembrane sequence, which anchors the α chain into the cell membrane of an antigen-presenting cell. MHC class I complexes, when associated with antigen (and in the presence of appropriate co-stimulatory signals), stimulate CD8+ cytotoxic T cells, which function to kill any cell which they specifically recognize.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy. 22^{nd} ed., London, UK: Pharmaceutical Press, 2013) describes compositions and formulations suitable for pharmaceutical delivery of the polypeptides, antibodies and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Photoimmunotherapy: A targeted therapy that utilizes an antigen-specific antibody-photoabsorber conjugate that can be activated by near-infrared light to kill targeted cells. The photon absorber is typically based on phthalocyanine dye, such as a near infrared (NIR) phthalocyanine dye (for example, IRDye®) 700DX, also know known as IR700). The antibody binds to the appropriate antigen (e.g., an E6-MHC complex) and the photo-activatable dye induces lethal damage to cell membranes after NIR-light exposure. NIR-light exposure (e.g., 690 nm) induces highly selective, necrotic cell death within minutes without damage to adjoining cells (see, for example, U.S. Application No. 2018/0236076). Thus, such methods can be used to kill cells infected with HPV, such as using the antibodies provided herein.

Polypeptide: A polymer in which the monomers are amino acid residues joined together through amide bonds. When the amino acids are alpha-amino acids, either the L-optical isomer or the D-optical isomer can be used. The terms “polypeptide” and “protein” are used herein interchangeably and include standard amino acid sequences as well as modified sequences, such as glycoproteins. The term “polypeptide” is specifically intended to cover naturally occurring proteins, as well as proteins that are recombinantly or synthetically produced. In the context of the present disclosure, a “polypeptide” is any protein or polypeptide (natural, recombinant or synthetic) that is capable of specific binding to a target antigen, such as an E6-MHC or E7-MHC complex. Thus, the polypeptides disclosed herein include at least one, such as one, two or three CDR sequences that mediate specific binding to the target antigen. In some aspects, the polypeptide is a single-domain monoclonal antibody, such as a camel single-domain monoclonal antibody, isolated from a phage display library, or a modified form thereof (such as a humanized or chimeric single-domain monoclonal antibody). In other aspects, the polypeptide comprises fibronectin (adectin), albumin, protein A (affibody), a peptide aptamer, an affimer, an affitin, an anticalin, or another antibody mimetic (see, e.g., Yu et al., Annu Rev Anal Chem 10(1): 293-320, 2017; Ta and McNaughton, Future Med Chem 9(12): 1301-1304, 2017; Koutsoumpeli et al., Anal Chem 89(5): 3051-3058, 2017), or a similar protein in which one or more CDR sequences (and/or HV sequences) have been incorporated to confer specific binding to the target antigen.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in HPV-infected cells and/or HPV-associated cancer cells. “Ameliorating” refers to the reduction in the number or severity of signs or symptoms of a disease, such as an HPV infection or an HPV-associated cancer.

Purified: The term purified does not require absolute purity; rather, it is intended as a relative term. Thus, for example, a purified peptide preparation is one in which the peptide or protein is more enriched than the peptide or protein is in its environment within a cell. In one aspect, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95%, 98%, 99%, 99.9% or 99.99% pure. Thus, in one specific, non-limiting example, a substantially purified protein is 90% free of other proteins or cellular components.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques.

Sample (or biological sample): A biological specimen containing genomic DNA, RNA (including mRNA), protein, or combinations thereof, which can be obtained from a subject or the environment. Examples include, but are not limited to, sputum, saliva, mucus, nasal wash, peripheral blood, tissue, cells, urine, tissue biopsy, fine needle aspirate, surgical specimen, feces, cerebral spinal fluid (CSF), bronchoalveolar lavage (BAL) fluid, Pap smear, nasopharyngeal samples, oropharyngeal samples, and autopsy material.

Sequence identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide or nucleic acid molecule will possess a relatively high degree of sequence identity when aligned using standard methods.

Methods of alignment of sequences for comparison are known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of an antibody that specifically binds an E6-MHC or E7-MHC complex are typically characterized by possession of at least about 75%, for example at least about 80%, 90%, 95%, 96%, 97%, 98% or 99% sequence identity counted over the full-length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Small molecule: A molecule, typically with a molecular weight less than about 1000 Daltons, or in some aspects, less than about 500 Daltons, wherein the molecule is capable of modulating, to some measurable extent, an activity of a target molecule.

Soluble T cell receptor: An engineered, soluble form of the T cell receptor (TCR) that lacks a transmembrane domain (see, e.g., Robinson et al., FEBS J 288:6159-6173, 2021; and Walseng et al., PLoS ONE 10(4): e0119559). Soluble TCRs can be used, for example, to target specific peptides (such as oncogenic peptides) presented in the context of MHC Class I. In some aspects described herein, the soluble TCR includes a polypeptide (such as a single-domain monoclonal antibody) that specifically recognizes HPV E6 peptide-MHC complexes or HPV E7 peptide-MHC complexes. The soluble TCR can further include, for example, an extracellular domain of a TCR alpha chain constant region and an extracellular domain of a TCR beta chain constant region; or an extracellular domain of a TCR gamma chain constant region and an extracellular domain of a TCR delta chain constant region.

Subject: Living multi-cellular vertebrate organisms, a category that includes both human and non-human animals (such as veterinary subjects or wild animals), for example birds, pigs, mice, rats, rabbits, sheep, horses, cows, dogs, cats, ferrets, deer, otters, bank voles, racoon dogs, tree shrews, fruit bats, hamsters, mink, and non-human primates (e.g., rhesus macaques, cynomolgus macaques, baboons, grivets and common marmosets). In one example, a subject is infected with HPV. In one example, a subject has an HPV associated cancer or precancerous lesion.

Synthetic: Produced by artificial means in a laboratory, for example a synthetic nucleic acid or protein (for example, an antibody) can be chemically synthesized in a laboratory.

Therapeutically effective amount: The amount of agent, such as a polypeptide (e.g., a single-domain monoclonal antibody) that is alone (or in combination with other therapeutic agents) sufficient to prevent, treat (including prophylaxis), reduce and/or ameliorate the symptoms and/or underlying causes of a disease or disorder, for example to prevent, inhibit, and/or treat an HPV infection or an HPV-associated cancer or pre-cancerous lesion. In some aspects, a therapeutically effective amount is sufficient to reduce or eliminate a symptom of a disease, such as an HPV-associated cancer or pre-cancerous lesion.

For instance, this can be the amount necessary to inhibit or suppress growth of a tumor or a pre-cancerous lesion. In one aspect, a therapeutically effective amount is the amount necessary to eliminate, reduce the size, or prevent metastasis of an HPV-associated cancer, such as reduce a tumor size and/or volume by at least 10%, at least 20%, at least 50%, at least 75%, at least 80%, at least 90%, at least 95%, or even 100%, and/or reduce the number and/or size/volume of metastases by at least 10%, at least 20%, at least 50%, at least 75%, at least 80%, at least 90%, at least 95%, or even 100%, for example as compared to a size/volume/number prior to treatment (or for example as compared to another treatment). In another aspect, a therapeutically effective amount is the amount necessary to reduce or eliminate the number or size of HPV-associated pre-cancerous lesions. When administered to a subject, a dosage will generally be used that will achieve target tissue concentrations (for example, in tumors) that has been shown to achieve a desired in vitro effect.

A therapeutically effective amount of an agent can be administered in a single dose, or in several doses, for example daily, during a course of treatment. However, the therapeutically effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Toxin: A molecule that is cytotoxic for a cell. Toxins include abrin, ricin, Pseudomonas exotoxin (PE), diphtheria toxin (DT), botulinum toxin, saporin, restrictocin or gelonin, or modified toxins thereof. For example, PE and DT are highly toxic compounds that typically bring about death through liver toxicity. PE and DT, however, can be modified into a form for use as an immunotoxin by removing the native targeting component of the toxin (such as domain Ia of PE or the B chain of DT) and replacing it with a different targeting moiety, such as an antibody.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements. In some aspects, the vector is a virus vector, such as a lentivirus vector, an adenovirus vector, or an adeno-associated virus (AAV). In some aspects, the vector is a plasmid.

### III. Polypeptides that Specifically Bind E6-MHC or E7-MHC Complexes

Typical 8-14mer peptides presented on MHC class I molecules include only about 2-3% of the total amino acids in a peptide-MHC (pMHC) complex and the peptide is spatially confined within the adjacent a-helices of the MHC groove (Hansen et al., Trends Immunol 31(10):363-369, 2010). HPV oncogenic peptide epitopes are difficult to reach by antibodies and thus pose a challenge for antibody development. Single-domain antibodies including the antigen binding variable domains of the shark immunoglobulin new antigen receptor (V_{NAR}) and the camelid variable region of the heavy chain (V_{H}H) (Muyldermans, Annu Rev Anim Biosci 9:401-421, 2021; Muyldermans, Annu Rev Biochem 82:775-797, 2013; English et al., Antib Ther 3(1):1-9, 2020) are suitable for development of TCR mimic (TCRm) antibodies because they are much smaller (approximately 15 kDa) than conventional antibodies and can target hidden or difficult epitopes (Muyldermans, Annu Rev Anim Biosci 9:401-421, 2021; Muyldermans, Annu Rev Biochem 82:775-797, 2013) like pMHC complexes.

Described herein are single-domain monoclonal antibodies (also called “nanobodies”), selected from camel V_{H}H phage libraries, that specifically bind HPV E6 peptide-MHC or HPV E7 peptide-MHC complexes. Nanobodies F5 and G9 bind E6-MHC complexes and nanobodies H3, H7 and H9 bind E7-MHC complexes. As described in the Examples, nanobodies F5 and G9 specifically recognize E6-MHC complexes in both the protein form and as expressed on cells. F5 CAR-T cells showed specific killing of target cells in vitro and inhibited the growth of HPV-associated tumors in a mouse model. The disclosed antibodies can be used, for example, in the treatment of HPV-associated cancers or pre-cancerous lesions, for the detection of HPV-infected cells and/or in methods of diagnosing a subject as having an HPV infection.

The amino acid sequences of the five disclosed nanobodies are provided below. Camel V_{H}H antibodies are comprised of the following regions (N-terminal to C-terminal): FR1-CDR1-FR2-CDR2-FR3-CDR3-FR4. Camel V_{H}H CDR residues were determined according to IMGT (italics), Kabat (underlined) and Paratome (bold). The amino acid positions of each CDR1, CDR2 and CDR3 are listed in Table 1.

F5

(SEQ ID NO: 1)
EVQLVESGGGSVQAGGSLRLSCAASGFTFSNNRMGWVRQAPGKGLE
WVSDINSSGGVTEYADSVKGRFTISRDNAKNTLYLQLNSLKIEDTA
SSIYYCVYYSYLMRLRPGQGTQVTV
G9
(SEQ ID NO: 2)
EVQLVESGGGLVQPGGSLRLSCVASGFTFSNNRMGWVRQAPGKGLE
WVSDINSSGGVTEYADSVKGRFTISRDNAKNTLYLQLNSLKIEDTA
IYYCVYYSYLMRLRPGQGTQVTVSS
H3
(SEQ ID NO: 3)
AVQLVESGGGLVQPGGSLRLSCAASGFTFSAFAMNWLRQAPGKGLE
WVAGISGTPSTYYADSVKGRFTISRDNAKSTLYLQLNSLKTEDTGM
YYCSQDRSYFVSVGQIGGTRGQGTQVTVSS
H7
(SEQ ID NO: 4)
QVQLVESGGGSVQAGGSLRLSCASSGFTFSAFAMNWLRQAPGKGLE
WVAGISGTPSTYYADSVKGRFTISRDNAKSTLYLQLNSLKTEDTGM
YYCSQDRSYFVSVGQIGGTRGQGTQVTVSS
H9
(SEQ ID NO: 5)
EVQLVESGGGLVQPGGSLRLSCAASGFTFSAFAMNWLRQAPGKGLE
WVAGISGTPSTYYADSVKGRFTISRDNAKSTLYLQLNSLKTEDTGM
YYCSQDRSYFVSVGQIGGTRGQGTQVTVSS

TABLE 1
Positions of the CDRs

| VHH | SEQ ID NO: | Scheme | CDR1 | CDR2 | CDR3 |
|---|---|---|---|---|---|
| F5 | 1 | IMGT | 26-33 | 51-58 | 97-106 |
| F5 | 1 | Kabat | 31-34 | 50-66 | 99-106 |
| F5 | 1 | Paratome | 27-35 | 47-60 | 97-107 |
| G9 | 2 | IMGT | 26-33 | 51-59 | 97-106 |
| G9 | 2 | Kabat | 31-34 | 50-66 | 99-106 |
| G9 | 2 | Paratome | 27-35 | 47-60 | 97-107 |
| H3 | 3 | IMGT | 26-33 | 51-57 | 96-111 |
| H3 | 3 | Kabat | 33-35 | 50-65 | 98-111 |
| H3 | 3 | Paratome | 27-35 | 47-59 | 97-112 |
| H7 | 4 | IMGT | 26-33 | 51-57 | 96-111 |
| H7 | 4 | Kabat | 33-35 | 50-65 | 98-111 |
| H7 | 4 | Paratome | 27-35 | 47-59 | 97-112 |
| H9 | 5 | IMGT | 26-33 | 51-57 | 96-111 |
| H9 | 5 | Kabat | 33-35 | 50-65 | 98-111 |
| H9 | 5 | Paratome | 27-35 | 47-59 | 97-112 |

Provided herein are polypeptides that bind (such as specifically bind) an HPV E6 or E7 peptide in complex with an MHC class I molecule. In some aspects, the polypeptide is a monoclonal antibody, for example a single-domain monoclonal antibody, such as a camel V_{H}H single-domain antibody. Also provided are compositions that include one or more of such antibodies, for example a composition that includes a pharmaceutically acceptable carrier.

In some aspects, the polypeptide (for example, single-domain monoclonal antibody) includes at least a portion of the amino acid sequence set forth herein as any one of SEQ ID NOs: 1-5, such as one or more (such as one, two or three) CDR sequence from any one of antibodies F5, G9, H3, H7 and H9, as determined using any CDR numbering scheme (such as IMGT, Kabat, Paratome or Chothia, or any combination thereof). In some examples, the polypeptide includes the CDR1, CDR2 and CDR3 sequences of any one of SEQ ID NOs: 1-5. In particular examples, the CDR sequences are determined using the Kabat, IMGT or Paratome numbering schemes, or a combination of the Kabat, IMGT and Paratome numbering schemes.

In some aspects, the polypeptide includes the CDR1, CDR2 and CDR3 sequences of nanobody F5 (SEQ ID NO: 1). In some examples, the CDR1, CDR2 and CDR3 sequences respectively include residues 26-33, 51-58 and 97-106 of SEQ ID NO: 1; residues 31-34, 50-66 and 99-106 of SEQ ID NO: 1; or residues 27-35, 47-60 and 97-107 of SEQ ID NO: 1. In particular examples, the amino acid sequence of the polypeptide is at least 80%, at least 85%, at least 90%, at least 95%, at least 96%, at least 97%, at least 98% or at least 99% identical to SEQ ID NO: 1. In specific examples, the amino acid sequence of the polypeptide includes or consists of SEQ ID NO: 1.

In some aspects, the polypeptide includes the CDR1, CDR2 and CDR3 sequences of nanobody G9 (SEQ ID NO: 2). In some examples, the CDR1, CDR2 and CDR3 sequences respectively include residues 26-33, 51-59 and 97-106 of SEQ ID NO: 2; residues 31-34, 50-66 and 99-106 of SEQ ID NO: 2; or residues 27-35, 47-60 and 97-107 of SEQ ID NO: 2. In particular examples, the amino acid sequence of the polypeptide is at least 80%, at least 85%, at least 90%, at least 95%, at least 96%, at least 97%, at least 98% or at least 99% identical to SEQ ID NO: 2. In specific examples, the amino acid sequence of the polypeptide includes or consists of SEQ ID NO: 2.

In some aspects, the polypeptide includes the CDR1, CDR2 and CDR3 sequences of nanobody H3 (SEQ ID NO: 3). In some examples, the CDR1, CDR2 and CDR3 sequences respectively include residues 26-33, 51-57 and 96-111 of SEQ ID NO: 3; residues 33-35, 50-65 and 98-111 of SEQ ID NO: 3; or residues 27-35, 47-59 and 97-112 of SEQ ID NO: 3. In particular examples, the amino acid sequence of the polypeptide is at least 80%, at least 85%, at least 90%, at least 95%, at least 96%, at least 97%, at least 98% or at least 99% identical to SEQ ID NO: 3. In specific examples, the amino acid sequence of the polypeptide includes or consists of SEQ ID NO: 3.

In some aspects, the polypeptide includes the CDR1, CDR2 and CDR3 sequences of nanobody H7 (SEQ ID NO: 4). In some examples, the CDR1, CDR2 and CDR3 sequences respectively include residues 26-33, 51-57 and 96-111 of SEQ ID NO: 4; residues 33-35, 50-65 and 98-111 of SEQ ID NO: 4; or residues 27-35, 47-59 and 97-112 of SEQ ID NO: 4. In particular examples, the amino acid sequence of the polypeptide is at least 80%, at least 85%, at least 90%, at least 95%, at least 96%, at least 97%, at least 98% or at least 99% identical to SEQ ID NO: 4. In specific examples, the amino acid sequence of the polypeptide includes or consists of SEQ ID NO: 4.

In some aspects, the polypeptide includes the CDR1, CDR2 and CDR3 sequences of nanobody H9 (SEQ ID NO: 5). In some examples, the CDR1, CDR2 and CDR3 sequences respectively include residues 26-33, 51-57 and 96-111 of SEQ ID NO: 5; residues 33-35, 50-65 and 98-111 of SEQ ID NO: 5; or residues 27-35, 47-59 and 97-112 of SEQ ID NO: 5. In particular examples, the amino acid sequence of the polypeptide is at least 80%, at least 85%, at least 90%, at least 95%, at least 96%, at least 97%, at least 98% or at least 99% identical to SEQ ID NO: 5. In specific examples, the amino acid sequence of the polypeptide includes or consists of SEQ ID NO: 5.

In some aspects, the polypeptide is a single-domain monoclonal antibody. In some examples, the single-domain monoclonal antibody is a camel V_{H}H single-domain antibody. In some examples, the single-domain monoclonal antibody is a humanized single-domain monoclonal antibody or a chimeric single-domain monoclonal antibody. In other examples, the polypeptide is a recombinant fibronectin or albumin. Further provided herein are polypeptide (for example, single-domain antibody) compositions that include at least two, at least three, at least four or at least five different polypeptides specific for E6-MHC or E7-MHC complexes as disclosed herein. In some aspects of the disclosed compositions, at least one polypeptide specifically binds an E6-MHC complex and at least one polypeptide specifically binds an E7-MHC complex. In some examples, the polypeptide compositions further include a pharmaceutically acceptable carrier, such as water or saline. In some examples, the polypeptide composition is lyophilized.

Also provided are fusion proteins that include an E6-MHC-specific or E7-MHC-specific polypeptide (for example, single-domain antibody) disclosed herein and a heterologous protein. In some aspects, the heterologous protein is an Fc protein or a leucine zipper. A single-domain antibody can be fused to an Fc region to generate a bivalent antibody (e.g., V_{H}H-Fc). In some examples, the Fc protein is a human Fc protein, such as the human IgG1 Fe. In particular non-limiting examples, the fusion protein includes a single-domain antibody disclosed herein, a hinge region and an Fc domain (such as the human IgG1 Fc domain). In one specific example, the fusion protein further includes a linker, such as an Ala-Ala-Ala linker located between the single-domain monoclonal antibody and the hinge region. In some aspects, provided is a disclosed single-domain monoclonal antibody in an IgG, IgA or IgM format.

Further provided herein are chimeric antigen receptors (CARs) that include a polypeptide (such as a single-domain monoclonal antibody) disclosed herein. In some aspects, the CAR further includes a hinge region, a transmembrane domain, a costimulatory signaling moiety, a signaling domain, or any combination thereof. In specific non-limiting examples, the hinge region includes a CD8a, CD28 or IgG4 hinge region; the transmembrane domain includes a CD8a or CD28 transmembrane domain; the costimulatory signaling moiety includes a 4-1BB signaling moiety, and/or the signaling domain includes a CD32 signaling domain.

Also provided herein are E6-MHC-specific or E7-MHC-specific polypeptides (for example, single-domain antibodies) modified to enable their use with a universal CAR system. In some aspects, the E6-MHC-specific or E7-MHC-specific polypeptide is fused to one component of a specific binding pair. In some examples, the polypeptide is fused to a leucine zipper or biotin.

Further provided are cells expressing an E6-MHC-specific or E7-MHC-specific CAR. In some examples, the cell is a T lymphocyte, such as a CTL, a natural killer (NK) cell, a B cells, a macrophage, a dendritic cell (DC) or an induced pluripotent stem cell. In some examples, the cells are allogeneic cells, such as allogeneic cells obtained from a healthy donor. In specific non-limiting examples, the cells, such as T cells, are genetically modified to express the CAR and optionally to disrupt expression of the endogenous TCR. CARs and CAR-expressing cells are further described in section IV.

Soluble T cell receptors (TCRs) that include a polypeptide (such as a single-domain monoclonal antibody) disclosed herein are also provided. In some examples, the soluble TCR further includes an extracellular domain of a TCR alpha chain constant region and an extracellular domain of a TCR beta chain constant region. In other examples, the soluble TCR further includes an extracellular domain of a TCR gamma chain constant region and an extracellular domain of a TCR delta chain constant region. In some instances, rather than replacing the variable regions of the TCRs, the CDR sequences of the variable regions of the soluble TCRs are replaced with the CDR sequences of a nanobody disclosed herein. Soluble TCRs are further described in section V.

Also provided herein are immunoconjugates that include a polypeptide (for example, a single-domain antibody) disclosed herein and an effector molecule. In some aspects, the effector molecule is a toxin, such as, but not limited to, Pseudomonas exotoxin or a variant thereof, such as PE38. In other aspects, the effector molecule is a detectable label, such as, but not limited to, a fluorophore, an enzyme or a radioisotope. In other aspects, the effector molecule is a photon absorber, such as IR700. Immunoconjugates that include a photon absorber can be used for photoimmunotherapy or in vivo diagnostic imaging. Immunoconjugates are further described in section VI.

Further provided herein are antibody-drug conjugates (ADCs) that include a drug conjugated to a polypeptide (for example, a single-domain antibody) disclosed herein. In some aspects, the drug is a small molecule, for example an anti-viral agent, an anti-microtubule agent, an anti-mitotic agent and/or a cytotoxic agent. ADCs are further described in section VII.

Also provided herein are multi-specific antibodies that include a polypeptide (for example, a single-domain antibody) disclosed herein and at least one additional monoclonal antibody or antigen-binding fragment thereof. In some aspects, the multi-specific antibody is a bispecific antibody. In other aspects, the multi-specific antibody is a trispecific antibody. Multi-specific antibodies are further described in section VIII.

Further provided herein are antibody-nanoparticle conjugates that include a nanoparticle conjugated to a polypeptide (for example, a single-domain antibody) disclosed herein. In some aspects, the nanoparticle includes a polymeric nanoparticle, nanosphere, nanocapsule, liposome, dendrimer, polymeric micelle, or niosome. In some aspects, the nanoparticle includes a cytotoxic agent or an anti-viral agent. In some examples, the anti-viral agent is cidofovir ((S)-1-(3-hydroxy-2-(phosphonomethoxy)-propyl) cytosine), biphenylsulphonacetic acid, a histone deacetylase (HDAC) inhibitor, a derivative of nordihydroguaiaretic acid (NDGA; such as tetra-O-methyl NDGA or tetra-acetyl NDGA), dihydroartemisinin (DHA; the major metabolite of the antimalarial drug artemisin), Roscovitine (also known as CYC202, Seliciclib and R-roscovitine), or a CCdk2 inhibitor. Antibody-nanoparticle conjugates are further described in section IX.

Further provided herein are nucleic acid molecules that encode a polypeptide, an antibody, a fusion protein, a single-domain monoclonal antibody in an IgG, IgA or IgM format, a CAR, a soluble TCR, an immunoconjugate, or a multiple-specific antibody disclosed herein. In some aspects, the nucleic acid molecule is operably linked to a promoter, such as an inducible or constitutive promoter. Vectors that include the disclosed nucleic acid molecules are also provided. In some examples, the vector is an expression vector. In other examples, the vector is a viral vector. Isolated cells that include a nucleic acid molecule are vector disclosed herein are further provided. In some examples, the isolated cell is a prokaryotic cell, such as an E. coli cell. In other examples, the isolated cell is a mammalian cell, such as a human cell. Nucleic acid molecules are further described in section X.

Compositions that include a pharmaceutically acceptable carrier and a polypeptide (for example, a single-domain monoclonal antibody), fusion protein (such as an Fc fusion, or nanobody in an IgG, IgA or IgM format), CAR, isolated cell (such as a CAR expressing cell, for example a CAR T cell, a CAR NK cell, a CAR B cells, a CAR DC, a CAR macrophage, or a CAR iPSC), soluble TCR, immunoconjugate, ADC, multi-specific antibody, antibody-nanoparticle conjugate, isolated nucleic acid molecule or vector disclosed herein are further provided by the present disclosure. In some examples, the composition includes a single-domain monoclonal antibody-Fc fusion protein (which forms a bivalent antibody). Compositions are further described in section XI.

Also provided herein are methods of treating an HPV-associated cancer or pre-cancerous lesion in the subject. In some aspects, the method includes administering to the subject a therapeutically effective amount of a polypeptide (for example, a single-domain monoclonal antibody), fusion protein (such as an Fc fusion, or nanobody in an IgG, IgA or IgM format), CAR, isolated cell (such as a CAR expressing cell, for example a CAR T cell, a CAR NK cell, a CAR B cells, a CAR DC, a CAR macrophage, or a CAR iPSC), soluble TCR, immunoconjugate, ADC, multi-specific antibody, antibody-nanoparticle conjugate, isolated nucleic acid molecule, vector or composition disclosed herein. In some examples, the HPV-associated cancer is a cervical cancer, vaginal cancer, vulvar cancer, penile cancer, anal cancer or oropharyngeal cancer (such as cancer in the soft palate, throat, tonsil, or back of tongue). In some examples, the method further includes administering a second anti-cancer therapy (such as chemotherapy, biological therapy (such as another mAb, such as one specific for EGFR (e.g., cetuximab), VEGF, CTLA4, PD-1 (e.g., nivolumab or pembrolizumab), or PD-L1), radiation therapy, surgical excision, cryosurgery, laser therapy and/or a checkpoint inhibitor) to the subject. Therapeutic methods are further described in section XII.

Further provided are methods of detecting an HPV-infected cell in a sample containing cells. In some aspects, the method includes contacting the sample with a polypeptide (such as a single-domain monoclonal antibody) disclosed herein and detecting binding of the polypeptide to cells in the sample, thereby detecting an HPV-infected cell. Also provided are methods of diagnosing a subject as having an HPV infection. In some aspects, the method includes contacting a sample containing cells obtained from the subject with a polypeptide (such as a single-domain monoclonal antibody) disclosed herein and detecting binding of the polypeptide to cells in the sample, thereby diagnosing the subject as having an HPV infection. In some examples of these methods, the polypeptide is directly labeled. In other examples, the method includes contacting the polypeptide with a detection antibody and detecting the binding of the detection antibody to the polypeptide, thereby detecting the HPV-infected cell in the sample or diagnosing the subject as having an HPV infection. In some examples, the sample is obtained from a subject, such as a human subject, suspected of having an HPV infection. In some examples, the HPV is HPV16 or HPV18. Diagnostic and detection methods are further described in section XIII.

Also provided are solid supports that include one or more of the HPV E6-MHC or E7-MHC specific polypeptides (such as single-domain monoclonal antibodies) disclosed herein. In some aspects, the solid support includes a bead, microchip, multiwell plate, or nitrocellulose having attached thereto one or more of the disclosed polypeptides (such as single-domain antibodies). The solid support can be used, for example, to detect HPV-infected cells.

### IV. Chimeric Antigen Receptors (CARs)

The disclosed polypeptides (such as single-domain antibodies) can be used to produce CARs and/or immune cells (such as T cells, natural killer (NK) cells, B cells, dendritic cells, or macrophages) or induced pluripotent stem cells (iPSCs) engineered to express CARs. In some aspects, CARs include a binding moiety, an extracellular hinge and spacer element, a transmembrane region and an endodomain that performs signaling functions (Cartellieri et al., J Biomed Biotechnol 2010:956304, 2010; Dai et al., J Natl Cancer Inst 108(7):djv439, 2016). In some instances, the binding moiety is an antigen binding fragment of a monoclonal antibody, such as a scFv, or a single-domain antibody (such as a camel V_{H}H antibody). The spacer/hinge region typically includes sequences from IgG subclasses, such as IgG1, IgG4, IgD, CD8, or CD28 domains. The transmembrane domain can be derived from a variety of different T cell proteins, such as CD3ζ, CD4, CD8, CD28 or inducible T cell co-stimulator (ICOS). Several different endodomains have been used to generate CARs. For example, the endodomain can consist of a signaling chain having an ITAM, such as CD3ζ or FcεRIγ. In some instances, the endodomain further includes the intracellular portion of at least one additional co-stimulatory domain, such as CD28, 4-1BB (CD137, TNFRSF9), OX-40 (CD134), CD30, ICOS, CD27, MYD88-CD40, killer cell immunoglobulin-like receptor 2DS2 (KIR2DS2) and/or DAP10.

In some aspects, the CAR is a two-chained antibody-T cell receptor (AbTCR), which includes the transmembrane and intracellular domains of the γ and δ chains (or alternatively, the α and β chains) of the TCR (see, e.g., Xu et al., Cell Discovery 4:62, 2018), wherein each of the γ and δ chains (or the α and β chains) is fused to an E6-MHC-specific or E7-MHC-specific single-domain antibody disclosed herein. When expressed in T cells, the AbTCR engages endogenous CD3 complexes to induce T cell activation (Xu et al., Cell Discovery 4:62, 2018).

In some aspects, the CAR is a synthetic T cell receptor and antigen receptor (STAR), which is a double-chain chimeric receptor that includes an antigen-binding domain (such an E6-MHC-specific or E7-MHC-specific monoclonal antibody) and the constant regions of the TCR that engage endogenous CD3 complexes (Liu et al., Sci Transl Med 13(586):eabb5191, 2021).

Immune cells (e.g., T cells, B cells, NK cells, DCs or macrophages) or iPSCs expressing CARs can be used to target a specific cell type, such as an HPV-infected cell. Thus, the polypeptides (such as single-domain antibodies) disclosed herein can be used to engineer immune cells or iPSCs that express a CAR containing the E6-MHC-specific or E7-MHC-specific monoclonal antibody, thereby targeting the engineered immune cells or iPSCs to cells infected with HPV and thereby expressing HPV E6/E7 proteins. In some aspects, the immune cells are autologous cells, such as immune cells isolated from a cancer patient, which are transfected/transformed with a nucleic acid molecule (e.g., a vector) encoding a CAR disclosed herein, and subsequently expanded ex vivo. In some examples, the autologous cells are infected with a viral vector (such as a lentiviral vector or an adeno-associated virus vector) encoding the CAR to produce the CAR-expressing immune cells. In other examples, non-viral transduction of autologous immune cells is performed via genome editing (such as by using CRISPR-Cas9) to produce the CAR-expressing immune cells (see, e.g., Zhang et al., Nature 609:369-374, 2022).

In other aspects, the immune cells are allogeneic immune cells, such as immune cells from peripheral blood or umbilical cord blood of a healthy donor (e.g., a donor that does not have cancer). Such allogenic immune cells can be manipulated as described above for autologous cells to express CARs (e.g., transformed/transfected/infected or subjected to genome editing). Since allogeneic cells can cause graft versus host disease (GVHD), steps can be taken to prevent or minimize GVDH (reviewed in Depil et al., Nat Rev 19:185-199, 2020). In some examples, the immune cells are from a donor who has received a stem cell transplant. In other examples, virus-specific memory T cells are the source of allogeneic cells. In yet other examples, non-αβ T cells are the source of allogeneic immune cells. In some instances, gene editing is used to prevent expression of a functional TCR at the surface of αβ T cells. For example, this can be achieved by disrupting the gene encoding the alpha chain of the T cell receptor.

In other aspects, a nucleic acid molecule or vector encoding a disclosed CAR is directly administered (e.g., injected) into a subject to generate CAR immune cells (such as CAR T cells) in vivo (reviewed in Wakao et al., Front Med 10:1141880, 2023). In some examples, the vector is a lentiviral vector, such as a lentiviral vector targeting a T cell marker (e.g., CD3, CD4 and/or CD8). In other examples, the vector is an adeno-associated virus (AAV) vector, such as an AAV targeting a T cell marker. Non-viral platforms can also be used to generate CAR immune cells in vivo, such as T cell targeted lipid nanoparticles or nanocarriers. The lipid nanoparticles or nanocarriers can be used to introduce nucleic acid, such as DNA or mRNA, encoding a disclosed CAR to generate in vivo CAR immune cells (such as CAR T cells).

Multispecific (such as bispecific) or bicistronic CARs are also contemplated by the present disclosure. In some aspects, the multispecific or bispecific CAR includes an antibody specific for E6-MHC complexes and an antibody specific for E7-MHC complexes. Similarly, a bicistronic CAR includes two CAR molecules expressed from the same construct where one CAR molecule is an E6-MHC-targeted CAR and the second CAR targets E7-MHC complexes. See, for example, Qin et al., Blood 130:810, 2017; and WO/2018/213337.

Accordingly, provided herein are CARs that include an E6-MHC-specific or E7-MHC-specific antibody, such as any one of the antibodies disclosed herein. Also provided are isolated nucleic acid molecules and vectors encoding the CARs (including bispecific and bicistronic CARs), and host cells, such as immune cells (e.g., T cells, B cells, NK cells, DCs, or macrophages) or induced pluripotent stem cells (iPSCs) expressing the CARs, bispecific CAR or bicistronic CARs. Immune cells or iPSCs expressing CARs comprised of an E6-MHC-specific or E7-MHC-specific monoclonal antibody can be used for the treatment of an HPV infection and/or treatment of an HPV-associated cancer or pre-cancerous lesion. In some aspects herein, the CAR is a bispecific CAR. In other aspects herein, the CAR is a bicistronic CAR.

In some aspects, the CAR includes a signal peptide sequence, for example, N-terminal to the antigen binding domain. The signal peptide sequence can be any suitable signal peptide sequence, such as a signal sequence from granulocyte-macrophage colony-stimulating factor receptor (GMCSFR), immunoglobulin light chain kappa, or IL-2. While the signal peptide sequence may facilitate expression of the CAR on the surface of the cell, the presence of the signal peptide sequence in an expressed CAR is not necessary for the CAR to function. Upon expression of the CAR on the cell surface, the signal peptide sequence may be cleaved off the CAR. Accordingly, in some aspects, the CAR lacks a signal peptide sequence.

In some aspects, the CARs disclosed herein are expressed from a construct (such as from a lentivirus or other viral vector) that also expresses a truncated version of human EGFR (huEGFRt). The CAR and huEGFRt are separated by a self-cleaving peptide sequence (such as T2A) such that upon expression in a transduced cell, the CAR is cleaved from huEGFRt (see, e.g., WO 2019/094482, which herein incorporated by reference).

The human epidermal growth factor receptor is comprised of four extracellular domains, a transmembrane domain and three intracellular domains. The EGFR domains are found in the following N-terminal to C-terminal order: Domain I-Domain II-Domain III-Domain IV-transmembrane (TM) domain-juxtamembrane domain-tyrosine kinase domain-C-terminal tail. Domain I and Domain III are leucine-rich domains that participate in ligand binding. Domain II and Domain IV are cysteine-rich domains and do not make contact with EGFR ligands. Domain II mediates formation of homo- or hetero-dimers with analogous domains from other EGFR family members, and Domain IV can form disulfide bonds with Domain II. The EGFR TM domain makes a single pass through the cell membrane and may play a role in protein dimerization. The intracellular domain includes the juxtamembrane domain, tyrosine kinase domain and C-terminal tail, which mediate EGFR signal transduction (Wee and Wang, Cancers 9(52), doi:10.3390/cancers9050052; Ferguson, Annu Rev Biophys 37:353-373, 2008; Wang et al., Blood 118(5):1255-1263, 2011).

A truncated version of human EGFR, referred to as “huEGFRt” includes only Domain III, Domain IV and the TM domain. Thus, huEGFRt lacks Domain I, Domain II, and all three intracellular domains. huEGFRt is not capable of binding EGF and lacks signaling activity. However, this molecule retains the capacity to bind particular EGFR-specific monoclonal antibodies, such as FDA-approved cetuximab (PCT Publication No. WO 2011/056894).

Transduction of immune cells (such as T cells, B cells, NK cells, DCs or macrophages) or iPSCs with a construct (such as a lentivirus vector) encoding both huEGFRt and an E6-MHC-specific or E7-MHC-specific CAR disclosed herein allows for selection of transduced cells using labelled EGFR monoclonal antibody cetuximab (ERBITUX™). For example, cetuximab can be labeled with biotin, and transduced immune cells or iPSCs can be selected using anti-biotin magnetic beads, which are commercially available (such as from Miltenyi Biotec). Co-expression of huEGFRt also allows for in vivo tracking of adoptively transferred CAR-expressing cells. Furthermore, binding of cetuximab to cells expressing huEGFRt induces cytotoxicity of ADCC effector cells, thereby providing a mechanism to eliminate transduced cells in vivo (Wang et al., Blood 118(5):1255-1263, 2011), such as at the conclusion of therapy.

Also provided herein are E6-MHC-specific and E7-MHC-specific polypeptides (such as a single-domain antibody disclosed herein) modified to enable their use with a universal CAR system. Universal CAR systems increase CAR flexibility and expand their use to additional antigens. Currently, for each patient who receives CAR T cell therapy, autologous T cells are cultured, expanded, and modified to express an antigen-specific CAR. This process is lengthy and expensive, limiting its use. Universal CARs are based on a system in which the signaling components of the CAR are split from the antigen-binding portion of the molecule but come together using a “lock-key” system. For example, biotin-binding immune receptor (BBIR) CARs are comprised of an intracellular T cell signaling domain fused to an extracellular domain comprising avidin. Biotinylated antigen-specific (such as E6-MHC-specific or E7-MHC-specific) monoclonal antibodies can then bind the BBIR to direct immune cells or iPSCs to E6- and/or E7-expressing cells. Another example is the split, universal and programmable (SUPRA) CAR system. In the SUPRA system, the CAR includes the intracellular signaling domains fused to an extracellular leucine zipper, which is paired with an antigen-specific monoclonal antibody fused to a cognate leucine zipper. For a review of universal CAR systems, see, for example, Zhao et al., J Hematol Oncol 11(1):132, 2018; and Cho et al., Cell 173:1426-1438, 2018. In some aspects herein, the E6-MHC-specific or E7-MHC-specific antibody is fused to one component of a specific binding pair. In some examples, the antibody is fused to a leucine zipper or biotin.

Another type of universal CAR can be generated using a sortase enzyme. A sortase is a prokaryotic enzyme that modifies surface proteins by recognizing and cleaving a carboxyl-terminal sorting signal. Sortase catalyzes transpeptidation between a sortase recognition motif and a sortase acceptor motif. Thus, antigen-specific CARs can be generated by contacting an antigen-specific antibody fused to a sortase recognition motif with a portion of a CAR molecule that includes the intracellular signaling domain(s), a transmembrane region and an extracellular portion comprising a sortase acceptor motif. In the presence of the sortase enzyme, the two components become covalently attached to form a complete antigen-specific CAR. Accordingly, in some aspects herein, an E6-MHC-specific or E7-MHC-specific antibody is modified to include a sortase recognition motif (see, for example, PCT Publication No. WO 2016/014553).

In some aspects, the universal CAR-T cell is a convertibleCAR™-T cell (see, e.g., Landgraf et al., Commun Biol 3(1):296, 2020). Using this universal format, the disclosed V_{H}H can be used as the antigen-recognition domain (the “MicAbody™”).

In some aspects, the E6-MHC-specific or E7-MHC-specific CAR is expressed in allogeneic immune cells (such as T cells, B cells, NK cells, DCs or macrophages), such as allogeneic immune cells from a healthy donor(s). In some examples, the allogeneic cells are genetically engineered to express the E6-MHC-specific or E7-MHC-specific CAR, for example by disrupting expression of an endogenous T cell receptor by insertion of the CAR (see, for example, MacLeod et al., Mol Ther 25(4):949-961, 2017). Gene editing can be performed using any appropriate gene editing system, such as CRISPR/Cas9, zinc finger nucleases or transcription activator-like effector nucleases (TALEN).

### V. Soluble T Cell Receptors

The polypeptides (such as single-domain antibodies) disclosed herein can also be used to produce soluble T cell receptors (TCRs) that specifically bind pMHC complexes. Soluble TCRs are engineered, soluble forms of a TCR that lacks a transmembrane domain (see, e.g., Robinson et al., FEBS J 288:6159-6173, 2021; and Walseng et al., PLoS ONE 10(4):e0119559; and US 2019/0336529). Soluble TCRs can be used, for example, to target specific peptides (such as oncogenic HPV peptides) presented in the context of MHC Class I.

Soluble TCRs generally include TCR variable regions (Vα and Vβ, or Vδ and Vγ) and extracellular domains of the TCR constant regions of the α/β or γ/δ chains but lack transmembrane and cytoplasmic domains of the constant regions.

In some aspects herein, a disclosed polypeptide (such as a V_{H}H single-domain antibody) replaces the α/β or γ/δ variable regions to confer specific binding to E6-MHC and/or E7-MHC complexes. In some examples, the soluble TCR includes two molecules of the same nanobody (one replacing each variable region), or includes two different nanobodies (such as one E6-MHC-specific nanobody and one E7-MHC-specific nanobody), each replacing one of the variable regions.

In other aspects, the native CDR sequences of the variable domains are replaced with the CDR sequences of a polypeptide (such as a single-domain antibody) disclosed herein to confer specific binding to E6-MHC and/or E7-MHC complexes. In some examples, the CDRs of both variable regions are replaced by the CDRs of the same nanobody. In other examples, the CDRs of each variable region are replaced with the CDRs of two different nanobodies (such as one E6-MHC-specific nanobody and one E7-MHC-specific nanobody).

Soluble TCRs are further described in, for example, U.S. Application Publication No. 2019/0336529. Methods of making soluble TCRs is described in, for example, U.S. Application Publication No. 2008/0153131.

### VI. Immunoconjugates

The disclosed polypeptides (such as single-domain monoclonal antibodies) can be conjugated to a therapeutic agent or effector molecule. Immunoconjugates include, but are not limited to, molecules in which there is a covalent linkage of a therapeutic agent to an antibody. A therapeutic agent is an agent with a particular biological activity directed against a particular target molecule or a cell bearing a target molecule. One of skill in the art will appreciate that therapeutic agents can include various drugs such as vinblastine, daunomycin and the like, cytotoxins such as native or modified Pseudomonas exotoxin or diphtheria toxin, encapsulating agents (such as liposomes) that contain pharmacological compositions, radioactive agents such as ¹²⁵I, ³²P, ¹⁴C, ³H and ³⁵S, photon absorbers such as IR700, and other labels, target moieties and ligands.

The choice of a particular therapeutic agent depends on the particular target molecule or cell, and the desired biological effect. Thus, for example, the therapeutic agent can be a cytotoxin that is used to bring about the death of a particular target cell (such as an HPV-infected cell). Conversely, where it is desired to invoke a non-lethal biological response, the therapeutic agent can be conjugated to a non-lethal pharmacological agent or a liposome containing a non-lethal pharmacological agent.

With the therapeutic agents and polypeptides (such as antibodies) described herein, one of skill can readily construct a variety of clones containing functionally equivalent nucleic acids, such as nucleic acids which differ in sequence, but which encode the same effector moiety or antibody sequence. Thus, the present disclosure provides nucleic acids encoding antibodies and conjugates and fusion proteins thereof.

Effector molecules can be linked to a polypeptide (such as an antibody) of interest using any number of means. Both covalent and noncovalent attachment means may be used. The procedure for attaching an effector molecule to an antibody varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of known linker molecules. The linker can be any molecule used to join the antibody to the effector molecule. The linker is capable of forming covalent bonds to both the antibody and to the effector molecule. Suitable linkers are well-known and include, but are not limited to, straight or branched-chain carbon linkers, heterocyclic carbon linkers, or peptide linkers. Where the antibody and the effector molecule are polypeptides, the linkers may be joined to the constituent amino acids through their side groups (such as through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates include linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site.

In view of the large number of methods that have been reported for attaching a variety of radiodiagnostic compounds, radiotherapeutic compounds, labels (such as enzymes or fluorescent molecules), drugs, toxins, and other agents to antibodies, a skilled person can determine a suitable method for attaching a given agent to an antibody or other polypeptide.

The polypeptides (such as antibodies) disclosed herein can be derivatized or linked to another molecule (such as another peptide or protein). In general, the antibodies or portion thereof is derivatized such that the binding to the target antigen is not affected adversely by the derivatization or labeling. For example, the antibody can be functionally linked (by chemical coupling, genetic fusion, noncovalent association or otherwise) to one or more other molecular entities, such as another antibody (for example, a bispecific antibody or a diabody), a detection agent, a photon absorber, a pharmaceutical agent, and/or a protein or peptide that can mediate association of the antibody or antibody portion with another molecule (such as a streptavidin core region or a polyhistidine tag).

One type of derivatized antibody is produced by cross-linking two or more antibodies (of the same type or of different types, such as to create bispecific antibodies). Suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate). Such linkers are commercially available.

A polypeptide (such as antibody) provided herein can be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP) and yellow fluorescent protein (YFP). An antibody can also be conjugated with enzymes that are useful for detection, such as horseradish peroxidase, B-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. An antibody or antigen binding fragment may also be conjugated with biotin and detected through indirect measurement of avidin or streptavidin binding. The avidin itself can also be conjugated with an enzyme or a fluorescent label.

An antibody provided herein may be labeled with a magnetic agent, such as gadolinium. Antibodies can also be labeled with lanthanides (such as europium and dysprosium), and manganese. Paramagnetic particles such as superparamagnetic iron oxide are also of use as labels. An antibody may also be labeled with a predetermined polypeptide epitopes recognized by a secondary reporter (such as leucine zipper pair sequences, binding sites for secondary antibodies, metal binding domains, epitope tags). In some aspects, labels are attached by spacer arms of various lengths to reduce potential steric hindrance.

An antibody provided herein can also be labeled with a radiolabeled amino acid. The radiolabel may be used for both diagnostic and therapeutic purposes. For instance, the radiolabel may be used to detect expression of a target antigen by x-ray, emission spectra, or other diagnostic techniques. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹In, ¹²⁵I, ¹³¹I.

An antibody disclosed herein can also be conjugated to a photon absorber. In some aspects, the photon absorber is a phthalocyanine dye, such as, but not limited to, IRDye® 700DX (also known as “IR700”). Antibody-photoabsorber conjugates can be used for photoimmunotherapy (for example to kill cells infected with HPV).

An antibody can also be derivatized with a chemical group such as polyethylene glycol (PEG), a methyl or ethyl group, or a carbohydrate group. These groups may be useful to improve the biological characteristics of the antibody, such as to increase serum half-life or to increase tissue binding.

Toxins can be employed with the monoclonal antibodies described herein to produce immunotoxins. Exemplary toxins include ricin, abrin, diphtheria toxin and subunits thereof, as well as botulinum toxins A through F. These toxins are readily available from commercial sources (for example, Sigma Chemical Company, St. Louis, MO). Contemplated toxins also include variants of the toxins described herein (see, for example, see, U.S. Pat. Nos. 5,079,163 and 4,689,401). In one aspect, the toxin is Pseudomonas exotoxin (PE) (U.S. Pat. No. 5,602,095). As used herein “Pseudomonas exotoxin” refers to a full-length native (naturally occurring) PE or a PE that has been modified. Such modifications can include, but are not limited to, elimination of domain Ia, various amino acid deletions in domains Ib, II and III, single amino acid substitutions and the addition of one or more sequences at the carboxyl terminus (for example, see Siegall et al., J. Biol. Chem. 264:14256-14261, 1989).

PE employed with the monoclonal antibodies described herein can include the native sequence, cytotoxic fragments of the native sequence, and conservatively modified variants of native PE and its cytotoxic fragments. Cytotoxic fragments of PE include those which are cytotoxic with or without subsequent proteolytic or other processing in the target cell. Cytotoxic fragments of PE include PE40, PE38, and PE35. For additional description of PE and variants thereof, see for example, U.S. Pat. Nos. 4,892,827; 5,512,658; 5,602,095; 5,608,039; 5,821,238; and 5,854,044; U.S. Patent Application Publication No. 2015/0099707; PCT Publication Nos. WO 99/51643 and WO 2014/052064; Pai et al., Proc. Natl. Acad. Sci. USA 88:3358-3362, 1991; Kondo et al., J. Biol. Chem. 263:9470-9475, 1988; Pastan et al., Biochim. Biophys. Acta 1333:C1-C6, 1997.

Also contemplated herein are protease-resistant PE variants and PE variants with reduced immunogenicity, such as, but not limited to PE-LR, PE-6X, PE-8X, PE-LR/6X and PE-LR/8X (see, for example, Weldon et al., Blood 113(16):3792-3800, 2009; Onda et al., Proc Natl Acad Sci USA 105(32):11311-11316, 2008; and PCT Publication Nos. WO 2007/016150, WO 2009/032954 and WO 2011/032022).

In some examples, the PE is a variant that is resistant to lysosomal degradation, such as PE-LR (Weldon et al., Blood 113(16):3792-3800, 2009; PCT Publication No. WO 2009/032954). In other examples, the PE is a variant designated PE-LR/6X (PCT Publication No. WO 2011/032022). In other examples, the PE variant is PE with reducing immunogenicity. In yet other examples, the PE is a variant designated PE-LR/8M (see PCT Publication No. WO 2011/032022).

Modification of PE may occur in any previously described variant, including cytotoxic fragments of PE (for example, PE38, PE-LR and PE-LR/8M). Modified PEs may include any substitution(s), such as for one or more amino acid residues within one or more T-cell epitopes and/or B cell epitopes of PE, or deletion of one or more T-cell and/or B-cell epitopes (see, for example, U.S. Patent Application Publication No. 2015/0099707).

Contemplated forms of PE also include deimmunized forms of PE, for example versions with domain II deleted (for example, PE24). Deimmunized forms of PE are described in, for example, PCT Publication Nos. WO 2005/052006, WO 2007/016150, WO 2007/014743, WO 2007/031741, WO 2009/32954, WO 2011/32022, WO 2012/154530, and WO 2012/170617.

The antibodies described herein can also be used to target any number of different diagnostic or therapeutic compounds to cells expressing E6-MHC or E7-MHC on their surface (e.g., HPV infected cells). Thus, an antibody of the present disclosure can be attached directly or via a linker to a drug that is to be delivered directly to cells expressing E6-MHC and/or E7-MHC. This can be done for therapeutic, diagnostic or research purposes. Therapeutic agents include such compounds as nucleic acids, proteins, peptides, amino acids or derivatives, glycoproteins, radioisotopes, photon absorbers, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides.

Alternatively, the molecule linked to an antibody can be an encapsulation system, such as a nanoparticle, liposome or micelle that contains a therapeutic composition such as a drug, a nucleic acid (for example, an antisense nucleic acid), or another therapeutic moiety that is preferably shielded from direct exposure to the circulatory system. Means of preparing liposomes attached to antibodies are well-known (see, for example, U.S. Pat. No. 4,957,735; Connor et al., Pharm. Ther. 28:341-365, 1985).

Antibodies described herein can also be covalently or non-covalently linked to a detectable label. Detectable labels suitable for such use include any composition detectable by spectroscopic, photochemical, biochemical, immunochemical, electrical, optical or chemical means. Useful labels include magnetic beads, fluorescent dyes (for example, fluorescein isothiocyanate, Texas red, rhodamine, green fluorescent protein, and the like), radiolabels (for example, ³H, ¹²⁵I, ³⁵S, ¹⁴C, or ³²P), enzymes (such as horseradish peroxidase, alkaline phosphatase and others commonly used in an ELISA), and colorimetric labels such as colloidal gold or colored glass or plastic (such as polystyrene, polypropylene, latex, and the like) beads.

Means of detecting such labels are known. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

### VII. Antibody-Drug Conjugates (ADCs)

ADCs are compounds comprised of an antigen-specific antibody (such as a single-domain antibody or antigen-binding fragment of an immunoglobulin) and a drug, for example an anti-viral agent (such as cidofovir ((S)-1-(3-hydroxy-2-(phosphonomethoxy)-propyl) cytosine), biphenylsulphonacetic acid, an HDAC inhibitor, a derivative of nordihydroguaiaretic acid (NDGA; such as tetra-O-methyl NDGA or tetra-acetyl NDGA), DHA, Roscovitine, a CCdk2 inhibitor, or another anti-viral agent used for the treatment of HPV) or a cytotoxic agent (such as an anti-microtubule agent or cross-linking agent). Because ADCs are capable of specifically targeting cells expressing a particular antigen, the drug can be much more potent than agents used for standard systemic therapy. For example, the most common cytotoxic drugs currently used with ADCs have an IC₅₀ that is 100- to 1000-fold more potent than conventional chemotherapeutic agents. Exemplary cytotoxic drugs include anti-microtubule agents, such as maytansinoids and auristatins (such as auristatin E and auristatin F). Other cytotoxins for use with ADCs include pyrrolobenzodiazepines (PBDs), which covalently bind the minor groove of DNA to form interstrand crosslinks. In some instances, ADCs include a 1:2 to 1:4 ratio of antibody provided herein to drug (Bander, Clinical Advances in Hematology & Oncology 10(8; suppl 10):3-7, 2012).

The antibody and drug can be linked by a cleavable or non-cleavable linker. However, in some instances, it is desirable to have a linker that is stable in the circulation to prevent systemic release of the cytotoxic drug that could result in significant off-target toxicity. Non-cleavable linkers prevent release of the cytotoxic agent before the ADC is internalized by the target cell. Once in the lysosome, digestion of the antibody by lysosomal proteases results in the release of the cytotoxic agent (Bander, Clinical Advances in Hematology & Oncology 10(8; suppl 10):3-7, 2012).

One method for site-specific and stable conjugation of a drug to a monoclonal antibody (or a V_{H}H-Fc protein) is via glycan engineering. Monoclonal antibodies have one conserved N-linked oligosaccharide chain at the Asn297 residue in the CH2 domain of each heavy chain (Qasba et al., Biotechnol Prog 24:520-526, 2008). Using a mutant β1,4-galactosyltransferase enzyme (Y289L-Gal-T1; U.S. Patent Application Publication Nos. 2007/0258986 and 2006/0084162), 2-keto-galactose is transferred to free GlcNAc residues on the antibody heavy chain to provide a chemical handle for conjugation.

The oligosaccharide chain attached to monoclonal antibodies can be classified into three groups based on the terminal galactose residues—fully galactosylated (two galactose residues; IgG-G2), one galactose residue (IgG-G1) or completely degalactosylated (IgG-G0). Treatment of a monoclonal antibody with β1,4-galactosidase converts the antibody to the IgG-G0 glycoform. The mutant β1,4-galactosyltransferase enzyme can transfer 2-keto-galactose or 2-azido-galactose from their respective UDP derivatives to the GlcNAc residues on the IgG-G1 and IgG-G0 glycoforms. The chemical handle on the transferred sugar enables conjugation of a variety of molecules to the monoclonal antibody via the glycan residues (Qasba et al., Biotechnol Prog 24:520-526, 2008).

Provided herein are ADCs that include a drug (such as an anti-viral agent) conjugated to a monoclonal antibody that binds (such as specifically binds) E6-MHC or E7-MHC complexes. In some aspects, the drug is a small molecule. In some examples, the drug is an anti-viral agent, such as cidofovir, biphenylsulphonacetic acid, an HDAC inhibitor, a derivative of NDGA (e.g., tetra-O-methyl NDGA or tetra-acetyl NDGA), DHA, Roscovitine, or a CCdk2 inhibitor. In some examples, the drug is a cross-linking agent, an anti-microtubule agent and/or anti-mitotic agent, or any cytotoxic agent suitable for mediating killing of tumor cells. Exemplary cytotoxic agents include, but are not limited to, a PBD, an auristatin, a maytansinoid, dolastatin, calicheamicin, nemorubicin and its derivatives, PNU-159682, anthracycline, vinca alkaloid, taxane, trichothecene, CC1065, camptothecin, elinafide, a combretastain, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity.

In some aspects, the ADC can further include a linker. In some examples, the linker is a bifunctional or multifunctional moiety that can be used to link one or more drug moieties to an antibody to form an ADC. In some aspects, ADCs are prepared using a linker having reactive functionalities for covalently attaching to the drug and to the antibody. For example, a cysteine thiol of an antibody can form a bond with a reactive functional group of a linker or a drug-linker intermediate to make an ADC.

In some examples, a linker has a functionality that is capable of reacting with a free cysteine present on an antibody to form a covalent bond. Exemplary linkers with such reactive functionalities include maleimide, haloacetamides, «-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates.

In some examples, a linker has a functionality that can react with an electrophilic group present on an antibody. Examples of such electrophilic groups include, but are not limited to, aldehyde and ketone carbonyl groups. In some cases, a heteroatom of the reactive functionality of the linker can react with an electrophilic group on an antibody and form a covalent bond to an antibody unit. Non-limiting examples include hydrazide, oxime, amino, hydrazine, thiosemicarbazone, hydrazine carboxylate and arylhydrazide.

In some examples, the linker is a cleavable linker, which facilitates release of the drug. Examples of cleavable linkers include acid-labile linkers (for example, comprising hydrazone), protease-sensitive linkers (for example, peptidase-sensitive), photolabile linkers, and disulfide-containing linkers (Chari et al., Cancer Res 52:127-131, 1992; U.S. Pat. No. 5,208,020).

The ADCs disclosed herein can be used for the treatment of an HPV infection or an HPV-associated cancer or pre-cancerous lesion, alone or in combination with another therapeutic agent and/or in combination with any standard therapy for the treatment of an HPV infection or an HPV-associated cancer or pre-cancerous lesion.

### VIII. Multi-Specific Antibodies

Multi-specific antibodies are recombinant proteins comprised of two or more monoclonal antibodies (such as single-domain antibodies) or antigen-binding fragments of two or more different monoclonal antibodies. For example, bispecific antibodies are comprised of two different monoclonal antibodies or antigen-binding fragments thereof. Thus, bispecific antibodies bind two different antigens or epitopes and trispecific antibodies bind three different antigens or epitopes. Multi-specific antibodies can be used for treating an HPV infection, or an HPV-associated cancer, by simultaneously targeting, for example, E6-MHC complexes and E7-MHC complexes. In some examples, the multi-specific antibody includes a first binding domain that targets E6-MHC complexes (such as a binding domain comprising nanobody F5 or G9) and a second binding domain that targets E7-MHC complexes (such as a binding domain comprising nanobody H3, H7 or H9).

In other aspects, the multi-specific antibodies include a monoclonal antibody that specifically binds E6-MHC or E7-MHC and an immune cell engager, for example, a T cell engager (e.g., an antibody that specifically binds CD3) or an NK cell engager (e.g., an antibody that specifically binds NKp46 or CD16). The E6-MHC-specific and E7-MHC-specific single-domain monoclonal antibodies disclosed herein can be used to generate multi-specific (such as bispecific or trispecific) antibodies that target both E6-MHC or E7-MHC expressing cells and CTLs, or target both E6-MHC or E7-MHC expressing cells and NK cells, thereby providing a means to treat HPV-infected cells or to treat an HPV-associated cancer or pre-cancerous lesion.

Bi-specific T-cell engagers (BiTEs) are a type of bispecific monoclonal antibody that are fusions of a first monoclonal antibody (such as a scFv or a single-domain antibody) that targets a specific antigen (such as E6-MHC or E7-MHC complexes) and a second antibody that binds T cells, such as CD3 on T cells.

Bi-specific killer cell engagers (BiKEs) are a type of bispecific monoclonal antibody that are fusions of a first monoclonal antibody (such as a scFv or single-domain antibody) that targets a specific antigen (such as E6-MHC or E7-MHC complexes) and a second scFv that binds a NK cell activating receptor, such as CD16.

Provided herein are multi-specific, such as trispecific or bispecific, monoclonal antibodies comprising an E6-MHC-specific or E7-MHC-specific monoclonal antibody. Also provided are isolated nucleic acid molecules and vectors encoding the multi-specific antibodies, and host cells comprising the nucleic acid molecules or vectors. Multi-specific antibodies comprising an E6-MHC-specific or E7-MHC-specific antibody can be used for the treatment of an HPV infection and/or an HPV-associated cancer or pre-cancerous lesion. Thus, provided herein are methods of treating a subject with an HPV infection, or a subject with an HPV-associated cancer or pre-cancerous lesion, by administering to the subject a therapeutically effective amount of an E6-MHC-targeting and/or E7-MHC-targeting multi-specific antibody.

### IX. Antibody-Nanoparticle Conjugates

The polypeptides (such as single-domain monoclonal antibodies) disclosed herein can be conjugated to a variety of different types of nanoparticles to deliver cytotoxic agents or anti-viral agents (such as cidofovir, biphenylsulphonacetic acid, an HDAC inhibitor, a derivative of NDGA (such as tetra-O-methyl NDGA or tetra-acetyl NDGA), DHA, Roscovitine, a CCdk2 inhibitor, or another agent used for the treatment of HPV infection) directly to HPV-infected cells via binding of the antibody to the E6-MHC or E7-MHC complexes expressed on the surface of infected cells. The use of nanoparticles reduces off-target side effects and can also improve drug bioavailability and reduce the dose of a drug required to achieve a therapeutic effect. Nanoparticle formulations can be tailored to suit the drug that is to be carried or encapsulated within the nanoparticle. For example, hydrophobic molecules can be incorporated inside the core of a nanoparticle, while hydrophilic drugs can be carried within an aqueous core protected by a polymeric or lipid shell. Examples of nanoparticles include, but are not limited to, nanospheres, nanocapsules, liposomes, dendrimers, polymeric micelles, niosomes, and polymeric nanoparticles (Fay and Scott, Immunotherapy 3(3):381-394, 2011).

Liposomes are common types of nanoparticles used for drug delivery. An antibody conjugated to a liposome is often referred to as an “immunoliposome.” The liposomal component of an immunoliposome is typically a lipid vesicle of one or more concentric phospholipid bilayers. In some cases, the phospholipids are composed of a hydrophilic head group and two hydrophobic chains to enable encapsulation of both hydrophobic and hydrophilic drugs. Conventional liposomes are rapidly removed from the circulation via macrophages of the reticuloendothelial system (RES). To generate long-circulating liposomes, the composition, size and charge of the liposome can be modulated. The surface of the liposome may also be modified, such as with a glycolipid or sialic acid. For example, the inclusion of polyethylene glycol (PEG) significantly increases circulation half-life. Liposomes for use as drug delivery agents, including for preparation of immunoliposomes, have been described in the art (see, for example, Paszko and Senge, Curr Med Chem 19(31)5239-5277, 2012; Immordino et al., Int J Nanomedicine 1(3):297-315, 2006; U.S. Patent Application Publication Nos. 2011/0268655; 2010/00329981).

Niosomes are non-ionic surfactant-based vesicles having a structure similar to liposomes. The membranes of niosomes are composed only of nonionic surfactants, such as polyglyceryl-alkyl ethers or N-palmitoylglucosamine. Niosomes range from small, unilamellar to large, multilamellar particles. These nanoparticles are monodisperse, water-soluble, chemically stable, have low toxicity, are biodegradable and non-immunogenic, and increase bioavailability of encapsulated drugs.

Dendrimers include a range of branched polymer complexes. These nanoparticles are water-soluble, biocompatible and are sufficiently non-immunogenic for human use. Generally, dendrimers consist of an initiator core, surrounded by a layer of a selected polymer that is grafted to the core, forming a branched macromolecular complex. Dendrimers are typically produced using polymers such as poly(amidoamine) or poly(L-lysine). Dendrimers have been used for a variety of therapeutic and diagnostic applications, including for the delivery of DNA, RNA, bioimaging contrast agents, chemotherapeutic agents and other drugs.

Polymeric micelles are composed of aggregates of amphiphilic co-polymers (consisting of both hydrophilic and hydrophobic monomer units) assembled into hydrophobic cores, surrounded by a corona of hydrophilic polymeric chains exposed to the aqueous environment. In many cases, the polymers used to prepare polymeric micelles are heterobifunctional copolymers composed of a hydrophilic block of PEG, poly(vinyl pyrrolidone) and hydrophobic poly(L-lactide) or poly(L-lysine) that forms the particle core. Polymeric micelles can be used to carry drugs that have poor solubility. These nanoparticles have been used to encapsulate a number of drugs, including doxorubicin and camptothecin. Cationic micelles have also been developed to carry DNA or RNA molecules.

Polymeric nanoparticles include both nanospheres and nanocapsules. Nanospheres consist of a solid matrix of polymer, while nanocapsules contain an aqueous core. The formulation selected typically depends on the solubility of the therapeutic agent to be carried/encapsulated; poorly water-soluble drugs are more readily encapsulated within nanospheres, while water-soluble and labile drugs, such as DNA and proteins, are more readily encapsulated within nanocapsules. The polymers used to produce these nanoparticles include, for example, poly(acrylamide), poly(ester), poly(alkylcyanoacrylates), poly(lactic acid) (PLA), poly(glycolic acids) (PGA), and poly(D,L-lactic-co-glycolic acid) (PLGA).

Antibodies provided herein can be conjugated to a suitable nanoparticle according to standard methods known in the art. For example, conjugation can be either covalent or non-covalent. In some aspects in which the nanoparticle is a liposome, the antibody is attached to a sterically stabilized, long circulation liposome via a PEG chain. Coupling of antibodies or antibody fragments to a liposome can also involve thioester bonds, for example by reaction of thiols and maleimide groups. Cross-linking agents can be used to create sulfhydryl groups for attachment of antibodies to nanoparticles (Paszko and Senge, Curr Med Chem 19(31)5239-5277, 2012).

### X. Nucleic Acid Molecules

Nucleic acid molecules (for example, DNA, cDNA, mRNA, or RNA molecules) encoding the amino acid sequences of the disclosed polypeptides, antibodies, fusion proteins, and conjugates that specifically bind to an E6-MHC or E7-MHC complex, are provided. Nucleic acid molecules encoding these molecules can readily be produced using the amino acid sequences provided herein (such as the CDR sequences and the V_{H}H sequences), sequences available in the art (such as framework or constant region sequences), and the genetic code. In some aspects, the nucleic acid molecules can be expressed in a host cell (such as a mammalian cell, yeast cell or a bacterial cell) to produce a disclosed polypeptide, antibody, fusion protein or antibody conjugate (e.g., CAR, soluble TCR, immunotoxin, multi-specific antibody).

Provided below are exemplary nucleic acid sequences of the disclosed camel V_{H}H antibodies specific for E6-MHC or E7-MHC complexes.

F5
(SEQ ID NO: 6)
GAGGTGCAGCTGGTGGAGTCTGGGGGAGGCTCGGTGCAGGCTGGA
GGGTCTCTGAGACTCTCCTGTGCAGCGTCTGGATTCACCTTCAGT
AACAATCGTATGGGCTGGGTCCGCCAGGCTCCAGGGAAGGGGCTC
GAGTGGGTCTCAGATATTAATAGTAGTGGTGGTGTCACTGAGTAT
GCAGACTCCGTGAAGGGCCGGTTCACCATCTCCAGAGACAACGCC
AAGAACACGCTGTATCTGCAATTGAACAGCCTGAAAATTGAGGAC
ACGGCCATCTATTACTGTGTTTATTATAGTTATCTCATGCGACTG
CGCCCGGGCCAGGGGACCCAGGTCACCGTCTCCTCA
G9
(SEQ ID NO: 7)
GAGGTGCAGCTGGTGGAGTCTGGGGGAGGCTTGGTGCAGCCCGGG
GGGTCTCTGAGACTCTCCTGTGTAGCCTCTGGATTCACCTTCAGT
AACAATCGTATGGGCTGGGTCCGCCAGGCTCCAGGGAAGGGGCTC
GAGTGGGTCTCAGATATTAATAGTAGTGGTGGTGTCACTGAGTAT
GCAGACTCCGTGAAGGGCCGGTTCACCATCTCCAGAGACAACGCC
AAGAACACGCTGTATCTGCAATTGAACAGCCTGAAAATTGAGGAC
ACGGCCATCTATTACTGTGTTTATTATAGTTATCTCATGCGACTG
CGCCCGGGCCAGGGGACCCAGGTCACCGTCTCCTCA
H3
(SEQ ID NO: 8)
GCGGTGCAGCTGGTGGAGTCTGGGGGAGGCTTGGTGCAGCCCGGG
GGGTCTCTGAGACTCTCCTGTGCAGCCTCTGGATTCACCTTCAGT
GCCTTTGCCATGAACTGGCTCCGCCAGGCTCCAGGGAAGGGGCTC
GAGTGGGTCGCAGGTATTAGTGGTACTCCCAGTACATACTATGCA
GACTCCGTGAAGGGCCGATTCACCATCTCCAGAGACAACGCCAAG
AGTACGCTGTATCTGCAATTGAACAGCCTGAAAACCGAAGACACG
GGCATGTATTACTGTTCGCAAGATCGTTCTTATTTTGTCTCGGTT
GGCCAAATCGGCGGGACGAGGGGCCAGGGGACCCAGGTCACCGTC
TCCTCA
H7
(SEQ ID NO: 9)
CAGGTGCAGCTGGTGGAGTCTGGGGGAGGCTCGGTGCAGGCTGGA
GGGTCCCTGAGACTCTCCTGTGCATCCTCTGGATTCACCTTCAGT
GCCTTTGCCATGAACTGGCTCCGCCAGGCTCCAGGGAAGGGGCTC
GAGTGGGTCGCAGGTATTAGTGGTACTCCCAGTACATACTATGCA
GACTCCGTGAAGGGCCGATTCACCATCTCCAGAGACAACGCCAAG
AGTACGCTGTATCTGCAATTGAACAGCCTGAAAACCGAAGACACG
GGCATGTATTACTGTTCGCAAGATCGTTCTTATTTTGTCTCGGTT
GGCCAAATCGGCGGGACGAGGGGCCAGGGGACCCAGGTCACCGTC
TCCTCA
H9
(SEQ ID NO: 10)
GAGGTGCAGCTGGTGGAGTCTGGGGGAGGCTTGGTGCAGCCCGGG
GGGTCTCTGAGACTCTCCTGTGCAGCCTCTGGATTCACCTTCAGT
GCCTTTGCCATGAACTGGCTCCGCCAGGCTCCAGGGAAGGGGCTC
GAGTGGGTCGCAGGTATTAGTGGTACTCCCAGTACATACTATGCA
GACTCCGTGAAGGGCCGATTCACCATCTCCAGAGACAACGCCAAG
AGTACGCTGTATCTGCAATTGAACAGCCTGAAAACCGAAGACACG
GGCATGTATTACTGTTCGCAAGATCGTTCTTATTTTGTCTCGGTT
GGCCAAATCGGCGGGACGAGGGGCCAGGGGACCCAGGTCACCGTC
TCCTCA

The genetic code can be used to construct a variety of functionally equivalent nucleic acid sequences, such as nucleic acids that differ in their sequence, but which encode the same antibody sequence, or encode a conjugate or fusion protein including the single-domain antibody sequence. In some aspects, the nucleic acid sequence is at least 80%, at least 85%, at least 90%, at least 95%, at least 96%, at least 97%, at least 98%, or at least 99% identical to any one of SEQ ID NOs: 6-10. In some examples, the nucleic acid sequence comprises or consists of any one of SEQ ID NOs: 6-10, or a degenerate variant thereof.

Nucleic acid molecules encoding the antibodies, fusion proteins, and conjugates that specifically bind to an E6-MHC or E7-MHC complex can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as in bacterial, plant, yeast, insect or mammalian cells. The antibodies and conjugates can be expressed as individual proteins including the single-domain monoclonal antibody (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011).

One or more DNA sequences encoding the antibodies, fusion proteins, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells, for example mammalian cells, such as the COS, CHO, HeLa and myeloma cell lines, can be used to express the disclosed antibodies and antigen binding fragments. Methods of stable transfer, meaning that the foreign DNA is continuously maintained in the host may be used.

The expression of nucleic acids encoding the antibodies and conjugates described herein can be achieved by operably linking the DNA or cDNA to a promoter (which is either constitutive or inducible), followed by incorporation into an expression cassette. The promoter can be any promoter of interest, such as a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, expression cassettes can include, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding an antibody or conjugate described herein without diminishing its biological activity. Some modifications can be made to facilitate the cloning, expression, or incorporation of the antibody into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the polypeptides, antibodies, fusion proteins, and conjugates can be purified according to standard procedures, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The polypeptides, antibodies, fusion proteins, and conjugates need not be 100% pure. Once purified, partially or to homogeneity as desired, if to be used prophylactically, the proteins should be substantially free of endotoxin.

Methods for expression of polypeptides, antibodies, fusion proteins, and conjugates, and/or refolding to an appropriate active form, from mammalian cells, and bacteria such as E. coli have been described and are applicable to the proteins disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989. The protein production of nanobodies such as camel V_{H}H has been described (Duan et al., Curr Protoc 2(6):e459, 2022).

### XI. Compositions

Compositions are provided that include one or more of the disclosed polypeptides (such as single-domain monoclonal antibodies) that bind (for example specifically bind) E6-MHC or E7-MHC in a carrier. Compositions that include fusion proteins (such as V_{H}H-Fc fusion proteins, or nanobodies in IgG, IgA or IgM format), ADCs, CARs (and immune cells and iPSCs expressing CARs), AbTCR, soluble TCR, multi-specific (such as bispecific or trispecific) antibodies, antibody-nanoparticle conjugates, immunoliposomes and immunoconjugates are also provided, as are nucleic acid molecule and vectors encoding the antibodies or antibody conjugates. The compositions can be prepared in unit dosage form for administration to a subject. The amount and timing of administration are at the discretion of the treating clinician to achieve the desired outcome. The polypeptide, antibody, fusion protein, ADC, CAR, CAR-expressing cell, AbTCR-expressing cell, soluble TCR, multi-specific antibody, antibody-nanoparticle conjugate, immunoliposome or immunoconjugate can be formulated for systemic or local administration. In one example, the composition is formulated for intravenous administration. In other examples, the composition is formulated for intramuscular administration or intraperitoneal administration. In other examples, the composition is formulated for vaginal administration. In other examples, the composition is formulated for rectal administration. In one example, the composition is lyophilized. In another example, the composition is formulated for oral administration. For example, a composition can include yeast or bacteria formulated to express a disclosed polypeptide (such as a single-domain monoclonal antibody).

In some aspects, the composition includes more than one E6-MHC-specific or E7-MHC-specific single-domain monoclonal antibody disclosed herein, such as 2, 3, 4 or 5 different antibodies. Kits are also provided that include one or more E6-MHC-specific or E7-MHC-specific single-domain monoclonal antibody disclosed herein, such as 2, 3, 4 or 5 different antibodies. Such kits can include one or more other therapeutic agents, such as those provided herein (e.g., other mAb, chemotherapeutic agent, anti-viral, or combinations thereof).

The compositions for administration can include a solution of the antibody, fusion protein, ADC, CAR, CAR-expressing cell (such as a T cell, NK cell, B cell, DC, macrophage or iPSC), soluble TCR, multi-specific (such as bispecific or trispecific) antibody, antibody-nanoparticle conjugate, immunoliposome or immunoconjugate in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody, conjugate, nucleic acid/vector or immune cell in these formulations can vary, and can be selected based on fluid volumes, viscosities, body weight and the like in accordance with the particular mode of administration selected and the subject's needs.

An exemplary pharmaceutical composition for intravenous administration includes about 0.1 to 10 mg of antibody (or fusion protein, ADC, CAR, soluble TCR, multi-specific antibody, antibody-nanoparticle conjugate, or immunoconjugate) per subject per day. Dosages from 0.1 up to about 100 mg per subject per day may be used, particularly if the agent is administered to a secluded site and not into the circulatory or lymph system, such as into a body cavity or into a lumen of an organ. In some aspects, the composition can be a liquid formulation including one or more antibodies in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml. Actual methods for preparing administrable compositions will be known or apparent to those skilled in the art and are described in more detail in such publications as Remington: The Science and Practice of Pharmacy, The University of the Sciences in Philadelphia, Editor, Lippincott, Williams, & Wilkins, Philadelphia, PA, 21^{st} Edition (2005).

The antibodies and conjugates disclosed herein can also be administered by other routes, including via inhalation or orally, such as by oral administration of yeast or bacteria (e.g., Lactococcus lactis) engineered to express a disclosed antibody or conjugate (see, e.g., Vandenbroucke et al., Mucosal Immunol 3(1):49-56, 2010).

Antibodies (or antibody conjugates, or nucleic acid molecules encoding such molecules) may be provided in lyophilized form and rehydrated with sterile water before administration, although they are also provided in sterile solutions of known concentration. The antibody solution can be added to an infusion bag containing 0.9% sodium chloride, USP, and in some cases administered at a dosage of from 0.5 to 15 mg/kg of body weight. Considerable experience is available in the art in the administration of antibody drugs, which have been marketed in the U.S. since the approval of RITUXAN™ in 1997. Antibodies, fusion proteins, ADCs, CARs (or CAR-expressing cells), soluble TCRs, multi-specific (such as bispecific or trispecific) antibodies, antibody-nanoparticle conjugates, immunoliposomes or immunoconjugates can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include, for example, microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the antibody-based compositions disclosed herein. Various degradable and nondegradable polymeric matrices for use in controlled drug delivery are known (Langer, Accounts Chem. Res. 26:537-542, 1993). For example, the block copolymer, polaxamer 407, exists as a viscous yet mobile liquid at low temperatures but forms a semisolid gel at body temperature. It is an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

Kits that include one or more disclosed compositions are provided. In some examples, a composition can be in a vial or other container. In some examples, the kit further includes an anti-viral agent, anti-cancer agent, or both, for example in separate vials. Exemplary anti-viral and anti-cancer agents that can be include in the kits are provided herein, such as Section XII below.

### XII. Methods of Treatment

Provided herein are methods of treating a human papillomavirus (HPV)-associated cancer or pre-cancerous lesion in a subject. The methods include administering to the subject a therapeutically effective amount of one or more polypeptides (such as single-domain antibodies) that specifically bind HPV E6-MHC or E7-MHC complexes, or a fusion protein, CAR, CAR-expressing cell (such as an immune cell or iPSC), soluble TCR, immunoconjugate, ADC, multi-specific antibody, antibody-nanoparticle conjugate, or composition disclosed herein.

In some aspects, the methods decrease the size, volume and/or weight of a tumor by at least 10%, at least 20%, at least 30%, at least 50%, at least 50%, at least 75%, at least 90%, at least 95%, at least 98%, at least 99% or 100%, for example relative to the size, volume and/or weight of the tumor prior to treatment. In some examples, the methods decrease the size, volume and/or weight of a metastasis by at least 10%, at least 20%, at least 30%, at least 50%, at least 50%, at least 75%, at least 90%, at least 95%, at least 98%, at least 99% or 100%, for example relative to the size, volume and/or weight of the metastasis prior to treatment. In some examples, the methods increase the survival time of a subject with an HPV-associated cancer by at least 3 months, at least 6 months, at least 9 months, at least 12 months, at least 18 months, at least 24 months, at last 36 months, at least 48 months, or at least 60 months, for example relative to the survival time in an absence of the treatment provided herein. In some examples, combinations of these effects are achieved.

In some aspects, the HPV-associated cancer is a cervical cancer, vaginal cancer, vulvar cancer, penile cancer, anal cancer or oropharyngeal cancer. In some aspects, the HPV-associated cancer is a cervical cancer. In some aspects, the HPV-associated cancer is an oropharyngeal cancer.

In some aspects, the method further includes administering a second anti-cancer therapy to the subject.

Administration of the polypeptides (such as single-domain antibodies), fusion proteins, CARs, CAR-expressing cells, soluble TCR, immunoconjugates, ADCs, multi-specific antibodies, antibody-nanoparticle conjugates, or compositions disclosed herein can also be accompanied by administration of other anti-cancer agents or therapeutic treatments. In some examples, the additional therapy includes chemotherapy, biological therapy (e.g., mAb), radiation therapy, surgical excision, cryosurgery, laser therapy and/or administration of a checkpoint inhibitor. In some examples, the additional therapy includes administration of an anti-viral agent (to reduce HPV in the subject), such as but not limited to, cidofovir, biphenylsulphonacetic acid, an HDAC inhibitor, a derivative of nordihydroguaiaretic acid (NDGA), for example tetra-O-methyl NDGA or tetra-acetyl NDGA, DHA, Roscovitine, or a CCdk2 inhibitor.

Any suitable anti-cancer agent can be administered in combination with the compositions disclosed herein. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g., anti-androgens) and anti-angiogenesis agents. Other anti-cancer treatments include radiation therapy and antibodies (e.g., mAbs) that specifically target cancer cells or other cells (e.g., anti-PD-1, anti-PD-L1, anti-CLTA4, anti-EGFR, or anti-VEGF). In one example, a cancer is treated by administering a polypeptide, antibody, fusion protein, CAR, CAR-expressing cell, immunoconjugate, ADC, multi-specific antibody, antibody-nanoparticle conjugate, or composition disclosed herein and one or more therapeutic mAbs, such as one or more of a PD-L1 antibody (e.g., durvalumab, KN035, cosibelimab, BMS-936559, BMS935559, MEDI-4736, MPDL-3280A, or MEDI-4737), anti-PD-1 antibody (e.g., pembrolizumab, cemiplimab, or nivolumab), anti-EGFR antibody (e.g., cetuximab or panitumumab), anti-VEGF antibody (e.g., bevacizumab or ramucizumab), or CLTA-4 antibody (e.g., ipilimumab or tremelimumab). In one example, a cancer is treated by administering an HPV E6-MHC-specific or E7-MHC-specific polypeptide (such as single-domain antibody) disclosed herein and one or more mAbs, for example: 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nivolumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, tisotumab vedotin-tftv, Trastuzumab, Tremelimumab, Tucotuzumab celmoleukin, Veltuzumab, Volociximab, Votumumab, Zalutumumab, or combinations thereof.

In one example, an E6-MHC-specific or E7-MHC-specific polypeptide (such as single-domain antibody) or a composition thereof is administered in combination with an alkylating agent, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

In one example, an E6-MHC-specific or E7-MHC-specific polypeptide (such as single-domain antibody) or a composition thereof is administered in combination with one or more antimetabolites, such as a folic acid analog (such as methotrexate), a pyrimidine analog (such as 5-FU or cytarabine), or a purine analog, such as mercaptopurine or thioguanine.

In one example, an E6-MHC-specific or E7-MHC-specific polypeptide (such as single-domain antibody) or a composition thereof is administered in combination with a natural product, such as a vinca alkaloid (such as vinblastine, vincristine, or vindesine), epipodophyllotoxin (such as etoposide or teniposide), antibiotic (such as dactinomycin, daunorubicin, doxorubicin, bleomycin, plicamycin, or mitomycin C), or enzyme (such as L-asparaginase).

In one example, an E6-MHC-specific or E7-MHC-specific polypeptide (such as single-domain antibody) or a composition thereof is administered in combination with a platinum coordination complex (such as cis-diamine-dichloroplatinum II also known as cisplatin), a substituted urea (such as hydroxyurea), a methyl hydrazine derivative (such as procarbazine), or an adrenocrotical suppressant (such as mitotane and aminoglutethimide).

In one example, an E6-MHC-specific or E7-MHC-specific polypeptide (such as single-domain antibody) or a composition thereof is administered in combination with a hormone and/or an antagonist, such as an adrenocorticosteroid (such as prednisone), a progestin (such as hydroxyprogesterone caproate, medroxyprogesterone acetate, and magestrol acetate), an estrogen (such as diethylstilbestrol and ethinyl estradiol), an antiestrogen (such as tamoxifen), or an androgen (such as testerone proprionate and fluoxymesterone).

In one example, an E6-MHC-specific or E7-MHC-specific polypeptide (such as single-domain antibody) or a composition thereof is administered in combination with a chemotherapeutic drug. Exemplary chemotherapy drugs that can be used in combination with the methods provided herein include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

In one example, an E6-MHC-specific or E7-MHC-specific polypeptide (such as single-domain antibody) or a composition thereof is administered in combination with an immunomodulator, such as AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, or TNF (tumor necrosis factor; Genentech). Further provided herein are methods of treating an HPV infection in a subject by administering to the subject a therapeutically effective amount of one or more polypeptides (such as single-domain antibodies) that specifically bind HPV E6-MHC or E7-MHC complexes, or a fusion protein, CAR, CAR-expressing cell (such as an immune cell of iPSC), soluble TCR, immunoconjugate, ADC, multi-specific antibody, antibody-nanoparticle conjugate, or composition disclosed herein. In some aspects, the therapeutically effective amount is an amount effective for reducing or inhibiting the HPV infection in the subject.

The HPV infection does not need to be completely eliminated or inhibited for the method to be effective. For example, the method can decrease the infection by at least 10%, at least 20%, at least 30%, at least 40%, at least 50%, at least 60%, at least 70%, at least 80%, at least 90%, at least 95%, at least 98%, at least 99%, or even 100% (elimination or prevention of detectable HPV infection) as compared to the HPV infection in the absence of the treatment.

In some aspects, an E6-MHC-specific or E7-MHC-specific polypeptide (such as single-domain antibody) or a composition thereof is administered in combination with one or more additional therapeutic agents.

In one example, an E6-MHC-specific or E7-MHC-specific polypeptide (such as single-domain antibody) or a composition thereof is administered in combination with (for example simultaneously, contemporaneously, or at separate times) one or more of bleomycin sulfate, cetuximab, docetaxel, cetuximab, hydrea (Hydroxyurea), Hydroxyurea, pembrolizumab, methotrexate sodium, nivolumab, onivolumab, pembrolizumab, taxotere (docetaxel), and trexall (methotrexate sodium), for example to treat an oropharyngeal cancer.

In one example, an E6-MHC-specific or E7-MHC-specific polypeptide (such as single-domain antibody) or a composition thereof is administered in combination with (for example simultaneously, contemporaneously, or at separate times) tisotumab vedotin-Tftv, for example to treat a cervical cancer.

In some aspects, an E6-MHC-specific or E7-MHC-specific polypeptide (such as single-domain antibody) or a composition thereof is administered in combination with (for example simultaneously, contemporaneously, or at separate times) an anti-viral agent. Exemplary anti-viral agents for the treatment of HPV include, but are not limited to, cidofovir, biphenylsulphonacetic acid, an HDAC inhibitor, a derivative of nordihydroguaiaretic acid (NDGA), for example tetra-O-methyl NDGA or tetra-acetyl NDGA, DHA, Roscovitine, or a CCdk2 inhibitor.

In some aspects, administration of a therapeutically effective amount of a disclosed polypeptide, antibody, fusion protein, soluble TCR, ADC, CAR, CAR-expressing cell (such as a T cell, B cell, NK cell, DC, macrophage or iPSC), multi-specific (such as bispecific or trispecific) antibody, antibody-nanoparticle conjugate, immunoconjugate, or composition inhibits the establishment of an infection and/or subsequent disease progression in a subject, which can encompass any statistically significant reduction in activity (for example, virus replication) or symptoms of the HPV infection in the subject (such as one or more HPV-associated cancers or pre-cancerous lesions).

Polypeptides, antibodies and conjugates thereof can be administered, for example, by intravenous infusion. Doses of the antibody or conjugate thereof can vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some aspects, the dose of the antibody or conjugate can be from about 0.5 mg/kg to about 5 mg/kg, such as a dose of about 1 mg/kg, about 2 mg/kg, about 3 mg/kg, about 4 mg/kg or about 5 mg/kg. The antibody or conjugate is administered according to a dosing schedule determined by a medical practitioner. In some examples, the antibody or conjugate is administered weekly, every two weeks, every three weeks or every four weeks.

### XIII. Diagnostic and Detection Methods

Methods are also provided for the detection of HPV-infected cells in vitro or in vivo. For example, the disclosed polypeptides (such as single-domain antibodies) and conjugates/compositions thereof can be used for in vivo imaging to detect an HPV infection. To use the disclosed polypeptides and antibodies as diagnostic reagents in vivo, the antibodies are labelled directly or indirectly with a detectable moiety, such as a radioisotope, fluorescent label, or positron emitting radionuclides. As one example, the polypeptides/antibodies disclosed herein can be conjugated to a positron emitting radionuclide for use in positron emission tomography (PET); this diagnostic process is often referred to as immunoPET. While full length antibodies can make good immunoPET agents, their biological half-life necessitates waiting several days prior to imaging, which increases associated non-target radiation doses. Smaller, single domain antibodies/nanobodies, such as those disclosed herein, have biological half-lives amenable to same day imaging.

In some examples, the presence of an HPV-infected cell is detected in a biological sample (containing cells) from a subject and can be used to identify a subject an HPV infection. In some examples, the HPV is a high-risk HPV, such as HPV16, HPV18, HPV31, HPV33, HPV34, HPV35, HPV39, HPV45, HPV51, HPV52, HPV56, HPV58, HPV59, HPV66, HPV68 or HPV70. The sample can be any sample from the subject that contains cells, such as a Pap smear, fecal sample, saliva sample, tissue biopsy or fine needle aspirate. The method of detection can include contacting a cell or sample, with an antibody or antibody conjugate (e.g., a conjugate including a detectable marker) that specifically binds to E6-MHC or E7-MHC complexes, under conditions sufficient to form an immune complex, and detecting the immune complex (e.g., by detecting a detectable marker conjugated to the antibody or antigen binding fragment). In some aspects, the subject from which the sample is obtained is a human subject. In other aspects, the subject from which the sample is obtained is a non-human animal subject, such as a non-human primate.

In one aspect, the polypeptide/antibody or antigen binding fragment is directly labeled with a detectable marker. In another aspect, the antibody is unlabeled and a secondary antibody or other molecule that can bind the primary antibody is utilized for detection. The secondary antibody can specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In one aspect, a kit is provided for detecting HPV-infected cells in a biological sample, such as a tissue biopsy or fine needle aspirate. Kits for detecting an HPV infection can include a monoclonal antibody that specifically binds E6-MHC or E7-MHC complexes, such as any of the nanobodies disclosed herein. In a further aspect, the antibody is labeled (for example, with a fluorescent, radioactive, or an enzymatic label). In some examples, such a kit includes a collection device for obtaining the sample, such as collection tubes/vials, syringes, swabs, and the like.

In one aspect, a kit includes instructional materials disclosing means of use of an antibody that binds E6-MHC or E7-MHC complexes. The instructional materials may be written, in an electronic form or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents used for the practice of a particular method.

In one aspect, the diagnostic kit comprises an immunoassay. Although the details of the immunoassays may vary with the particular format employed, the method of detecting E6-MHC or E7-MHC complexes in a biological sample generally includes the steps of contacting the biological sample (containing cells) with an antibody which specifically reacts, under immunologically reactive conditions, to E6-MHC or E7-MHC complexes. The antibody is allowed to specifically bind under immunologically reactive conditions to form an immune complex, and the presence of the immune complex (bound antibody) is detected directly or indirectly.

The antibodies disclosed herein can also be utilized in immunoassays, such as, but not limited to radioimmunoassays (RIAs), ELISA, lateral flow assay (LFA), or immunohistochemical assays. The antibodies can also be used for fluorescence activated cell sorting (FACS), such as for identifying/detecting virus-infected cells. FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies that bind E6-MHC or E7-MHC complexes, as disclosed herein, can be used in these assays. Thus, the antibodies can be used in a conventional immunoassay, including, without limitation, ELISA, RIA, LFA, FACS, tissue immunohistochemistry, Western blot or immunoprecipitation. The disclosed nanobodies can also be used in nanotechnology methods, such as microfluidic immunoassays, which can be used to capture HPV-infected cells. Suitable samples for use with a microfluidic immunoassay or other nanotechnology method, include but are not limited to, saliva, blood, cervical, and fecal samples. Microfluidic immunoassays are described in U.S. Patent Application No. 2017/0370921, 2018/0036727, 2018/0149647, 2018/0031549, 2015/0158026 and 2015/0198593; and in Lin et al., JALA June 2010, pages 254-274; Lin et al., Anal Chem 92:9454-9458, 2020; and Herr et al., Proc Natl Acad Sci USA 104(13): 5268-5273, 2007, all of which are herein incorporated by reference). Thus, in some examples, a kit of the present disclosure further includes secondary antibodies (which may be labeled), multiwell plates, nitrocellulose, FACS bead standards, and the like.

### EXAMPLES

The following examples are provided to illustrate particular features of certain aspects of the disclosure, but the scope of the claims should not be limited to those features exemplified.

### Example 1: Materials and Methods

This example provides the materials and methods for the studies described in Examples 2-6.

### Cells and Reagents

Cell lines were cultured in media consisting of RPMI 1640 (CaSki, T2) or DMEM (SCC90, 293 lines) supplemented with 10% FBS and 1% penicillin-streptomycin at 37° C. in a humidified atmosphere with 5% CO₂. Caski cells are an HLA-A*02:01+ HPV16+ cervical cancer cell line. SCC90 is an HLA-A*02:01+ HPV16+ squamous cell line from tumors of the head and neck. The 293E6 and 293E7 lines are 293-based lines with stable expression of HLA-A2 and E6 or E7, respectively. Peripheral blood mononuclear cells (PBMCs) were isolated from the blood of healthy donors using Ficoll (Cytiva) according to manufacturer's instructions. All cell lines were authenticated by morphology and growth rate and were mycoplasma free.

### Peptides and Monomers

Peptides were synthesized at a purity of >95% (Genscript, NJ). HLA: A*02:01 and beta-2 microglobulin (B2m) were expressed and purified from bacteria separately. These two molecules were mixed with synthesized peptides (E6 or E7; purity >95%) and folded to generate a peptide/MHC complex (Monomer): pMHC-E6, HLA-A*02:01-TIHDIILECV (SEQ ID NO: 11); pMHC-E7, HLA-A*02:01-YMLDLQPET (SEQ ID NO: 22). HLA-A2 was refolded with the peptide and B2m, purified by gel filtration, and biotinylated (NIH Tetramer Core at Emory University). The monomers were aliquoted and stored in-80° C. for further applications.

Phage Panning and ELISA Camel single domain antibody phage display libraries with a diversity greater than 10¹⁰ phage particles per milliliter were used for phage panning as described previously (Hong et al., Proc Natl Acad Sci USA 119(18):e2201433119, 2022). Briefly, the monomer (5 μg/ml) in PBS was coated on the immunotube at 4° C. overnight. Both the immunotube and the 1012 phages were blocked with 3% skimmed milk in PBS/Tween-20 (0.05%) for 1 hour at room temperature. Then pre-blocked phage supernatant was added to the tube to allow binding. After 1 hour of incubation at room temperature, the unbound and nonspecifically bound phages were removed using 10 washes with PBS/Tween-20 (0.05%) and 10 washes with PBS. The specifically bound phage was eluted with 500 μl 100 mM trimethylaminade for 15 minutes at room temperature. The eluate was neutralized with 250 μl of 1 M Tris-HCl buffer (pH 7.5) and was used to infect freshly prepared E. coli TG1 cells.

After four rounds of panning, 96 randomly picked clones were analyzed for antigen binding by monoclonal phage ELISA. Maxisorp 96-well plate (Fisher Scientific) was coated with the E6 complex or the control E7 complex. Phage ELISA followed previously described protocols (Ho et al., J Biol Chem 280(1): 607-617, 2005; Kim and Ho, Curr Protoc Protein Sci 94(1):e66, 2018) and results were read using a spectrophotometer (Molecular Devices) at 450 nm.

### Antibody Production and Purification

Soluble antibody protein was produced and purified as previously described (Duan et al., Curr Protoc 2(6):e459, 2022). Briefly, the pComb3x phagemids containing F5 or G9 sequences were transformed into HB2151 E. coli cells. The colonies were pooled and shaken in 1 L 2YT media containing 2% glucose, 100 μg/ml ampicillin at 37° C. until the OD600 reached 0.8 to 1. Fresh 2YT media containing 1 mM IPTG (Sigma) and 100 μg/ml ampicillin was added after the bacteria cells were spun down. The culture was shaken at 30° C. overnight for soluble protein production. The bacteria cells were spun down and lysed with polymyxin B (Sigma) for 1 hour at 37° C. to release the soluble protein. The supernatant was harvested after lysis and purified using the HisTrap column (Cytiva) using AKTA (GE Healthcare).

### Affinity Measurement by Octet

Binding kinetics was determined using the Octet RED96 system (FortéBio) at the Biophysics core at NHLBI, NIH (Bethesda, MD). F5 or G9 was immobilized onto NTA sensor tips. The antibody-coated tips were then dipped into PBS to stabilize the curve and then dipped into 25 nM E6 or E7 monomer for association then dipped into PBS for dissociation. Raw data was processed using Octet Data Analysis Software 9.0 to determine K_{D} value.

### FACS

The binding of F5 and G9 to the E6-MHC complex on the cell surface was detected by anti-Flag-APC-conjugated antibody. The transduction efficiencies of F5 CAR on T cells were detected by anti-EGFR human monoclonal antibody cetuximab (Erbitux) and goat-anti-human IgG-PE or allophycocyanin-conjugated antibody (Jackson ImmunoResearch). Data acquisition was performed using Sony A3800 (Sony) and analyzed using FlowJo software (TreeStar, Ashland).

### T2 Peptide Pulsing Assay

One million T2 cells were pulsed with the peptides at a concentration of 50 μM overnight at 37° C. Cells were then stained with either anti-HLA antibody (BB21, Invitrogen) for measuring the expression of the complex, or the nanobodies F5 or G9 and then anti-Flag-APC for the antibody binding. Samples were acquired using a Sony A3800 flow cytometer. Flow cytometry data files were analyzed with the FlowJo software.

### CAR T Production and Cell Killing

V_{H}H F5 was subcloned into the second-generation (2G) CAR construct, which contains expression cassettes encoding the CD8a hinge and transmembrane region, a 4-1BB costimulatory domain, the intracellular CD3ζ, the self-cleaving T2A sequence, and the truncated human epidermal growth factor receptor (EGFR) for cell tracking and ablation. The truncated human EGFR lacks the domains essential for ligand binding and tyrosine kinase activity but retains the binding epitope of anti-EGFR monoclonal antibody cetuximab. Recombinant F5-CAR lentiviral vectors were produced by co-transfecting with packaging plasmid psPAX2 and enveloping plasmid pMD2.G into HEK-293T cells using Calfectin (SignaGen, Rockville, MD). The psPAX2 and pMD2.G plasmids were from Addgene (#12260 and #12259). Lentiviral particles were collected from the supernatant 72 hours post-transfection and concentrated 100-fold by Lenti-X concentrator (Clontech, Mountain View, CA) in accordance with manufacturer's instructions. Peripheral blood mononuclear cells from healthy donors were stimulated for 24 hours using anti-CD3/anti-CD28 antibody-coated beads (Invitrogen, Carlsbad, CA) at a bead to cell ratio of 2:1 according to manufacturer's instructions in the presence of interleukin (IL)-2. To track T-cell numbers over time, viable cells were counted using trypan blue.

The cytolytic activity of T cells transduced with F5-CAR was determined by a luciferase-based assay as described previously (Li et al., Mol Ther Oncolytics 24:849-863, 2022). Briefly, CAR T cells and luciferase-expressing target cells (293 lines and Caski) were incubated for 24 hours at different effector to target ratios. The luciferase activity was measured using the luciferase assay system (Promega, Madison, WI) on a plate reader (PerkinElmer). The killing activity was normalized using mock T cells.

### Animal Studies

Five-week-old female NOD scid gamma (NSG) mice (NCI CCR Animal Resource Program/NCI Biological Testing Branch) were housed and treated under protocol LMB-059 approved by the Institutional Animal Care and Use Committee at the NIH. Tumors were initiated by subcutaneous injection of 1×10⁶ Caski tumor cells on the flank. Tumor treatment was on day 13 following tumor cell injection and consisted of a single intravenous injection of T cells (either 10 million CAR-T cells or untransduced T cells). Tumor size was determined by caliper measurement of the perpendicular diameters of each tumor and is reported as tumor area.

### Multiplex Cytokine Analysis

A human CD8/NK panel premixed bead-based multiplex cytokine assay from BioLegend can simultaneously detect the following cytokines by FACS: IL-2, IL-4, IL-6, IL-10, IL-17A, TNFα sFas, sFasL, IFNγ, Granzyme A, Granzyme B, Perforin and Granulysin. Supernatants were collected from cultures that were assigned for functional studies and stored at −80° C. until used for FACS. The data was analyzed according to the manufacturer's manual.

### Statistical Analysis

Statistical analyses were performed using Prism GraphPad software. For studies comparing two groups, a Student's t test was used. For studies with multiple groups, a one-way ANOVA followed by Dunnett test which accounts for multiple comparisons was used. All analyses were two-tailed. A p value of <0.05 was considered statistically significant.

### Example 2: Identification of Camel V_{H}H Nanobodies F5 and G9 from Phage Display

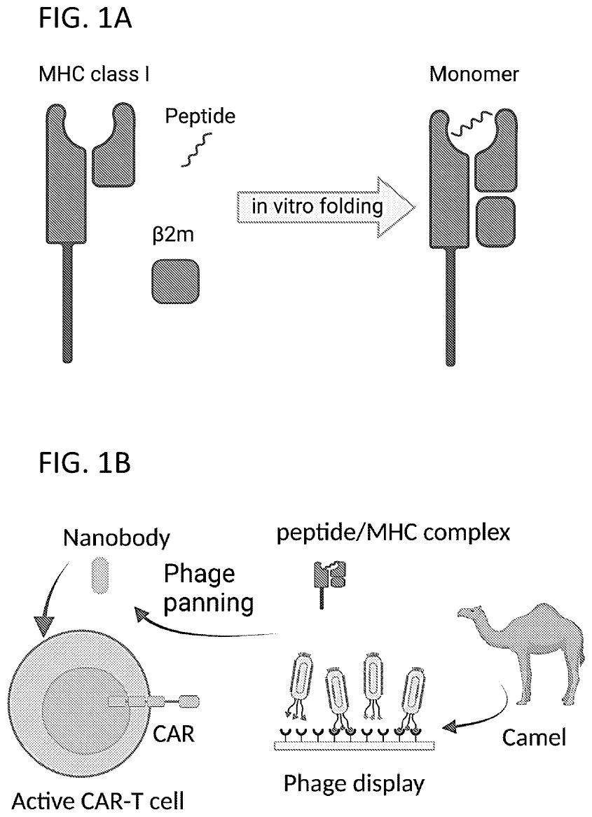

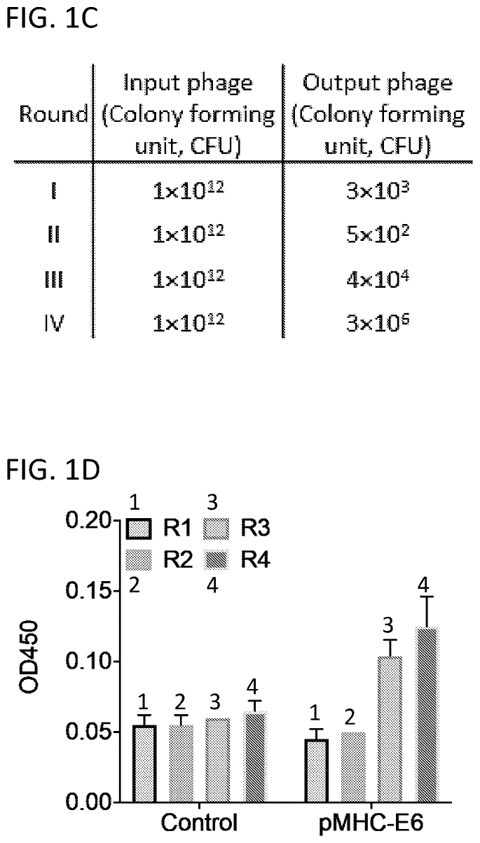

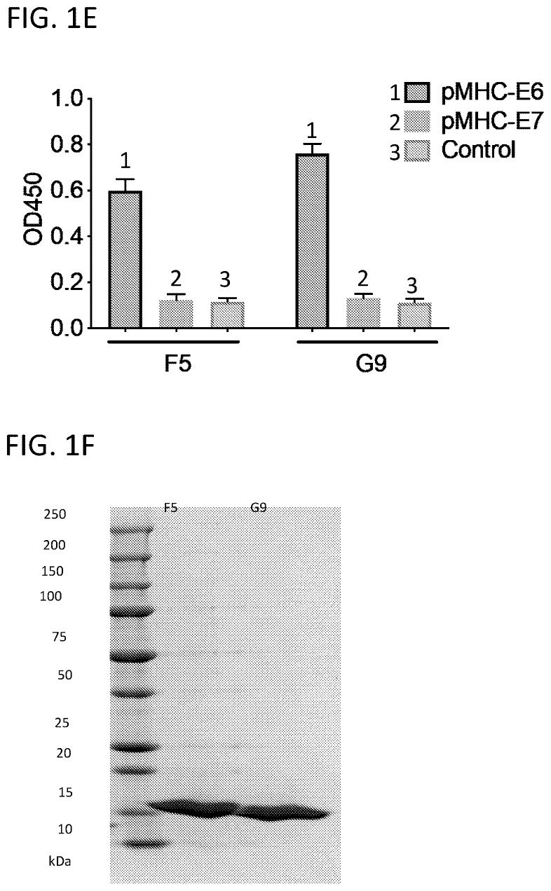

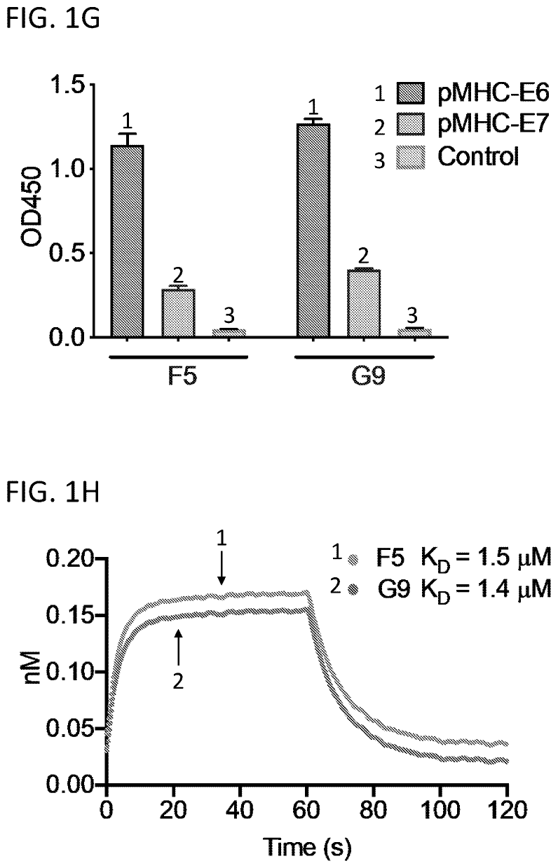

The monomers of E6 or E7 peptide and HLA-A*02:01 were synthesized (FIG. 1A). To identify nanobodies against the monomer, phage panning was carried out using camel single domain phage display libraries constructed from six camels, three males and three females, with ages ranging from 3 months to 20 years (FIG. 1B). After four rounds of panning, there was increased phage output (FIG. 1C) and about three-fold enrichment of eluted phage colonies by polyclonal phage ELISA (FIG. 1D). At the end of the fourth round of panning, 20 individual clones were identified to bind the E6 monomer protein by the monoclonal phage ELISA, and 8 unique binders were confirmed by subsequent sequencing. Two camel V_{H}Hs binders, F5 and G9, were identified that showed specific binding to the E6 monomer on monoclonal phage and protein ELISA (FIGS. 1E-1G). To measure the binding affinity against the E6 monomer, an Octet analysis was performed. The K_{D} of F5 and G9 were approximately 1 μm (FIG. 1H), which is similar to the affinity of most TCRs for binding to their target complexes (Campillo-Davo et al., Cells 9(7):1720, 2020).

### Example 3: Cell Binding of F5 and G9

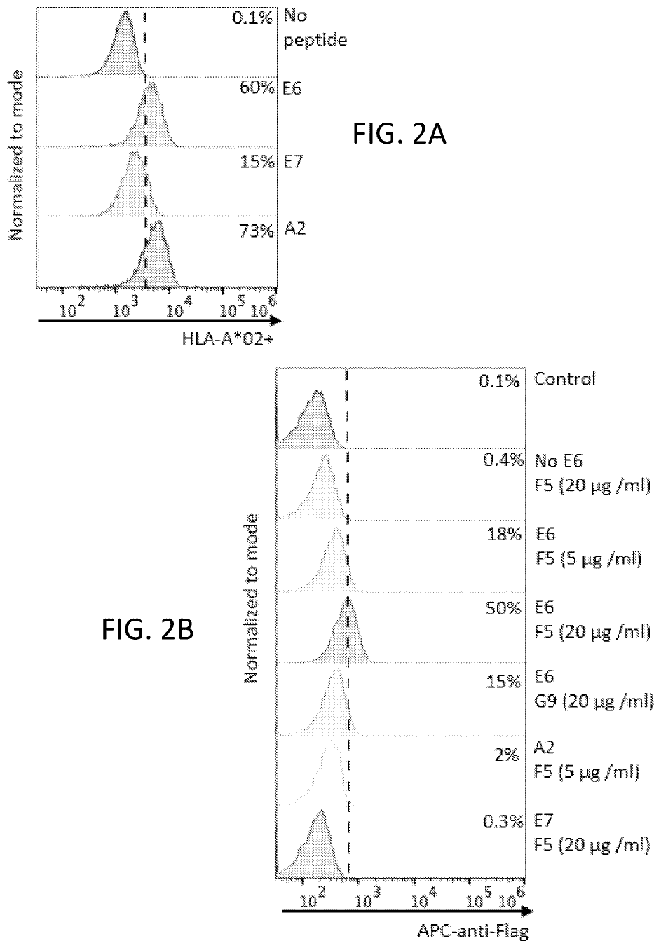

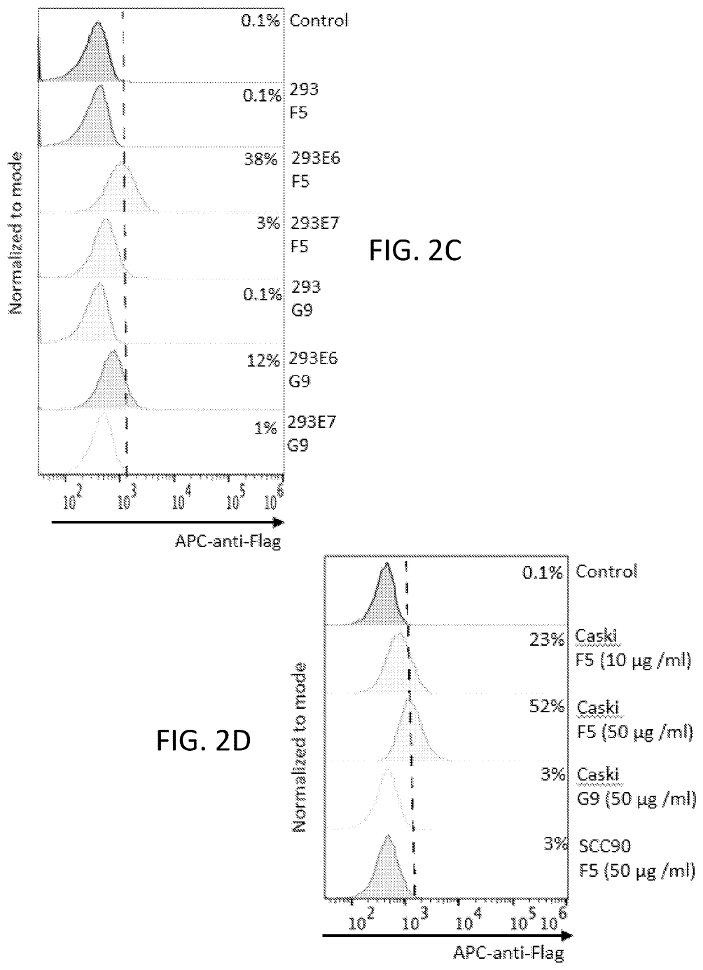

Antibody binding specificity for E6-MHC was further validated on the surface of human T2 lymphoblast cells. T2 cells are deficient in the transporter associated with antigen processing (TAP) and express unstable empty HLA molecules on the surface. The addition of the peptide and beta-2-microglobulin stabilizes the HLA molecule on the cell surface by forming a complex, which can be measured by FACS signal with an anti-HLA-A*02 antibody. The E6, E7 and A2 (influenza virus) peptides were pulsed on T2 cells successfully (FIG. 2A). F5 and G9 stained the cell surface of T2 cells pulsed with the target E6 peptide but not T2 cells pulsed with the E7 or A2 peptides (FIG. 2B). G9 had less binding to the complex compared with F5 at the same concentration (20 μg/ml) (15% vs 50%), suggesting weaker binding ability of G9.

To further validate the binding specificity of F5 and G9, 293 cells expressing E6 or E7 complex were used. F5 and G9 bound the 293E6 cell line more specifically over 293E7 and 293T cell controls. F5 showed higher levels of binding to 293E6 cells than G9 (FIG. 2C). Next, it was investigated whether F5 could recognize naturally processed E6-MHC on the surface of cancer cells. Among the HPV16+ cancer cell lines tested (Caski and SCC90), F5 showed high binding to Caski cells in a dose dependent manner (FIG. 2D). The binding on SCC90 was much lower than on Caski cells. Therefore, Caski cells were chosen for the following experiments.

### Example 4: The Residues of E6 Peptide Involved in F5 Binding

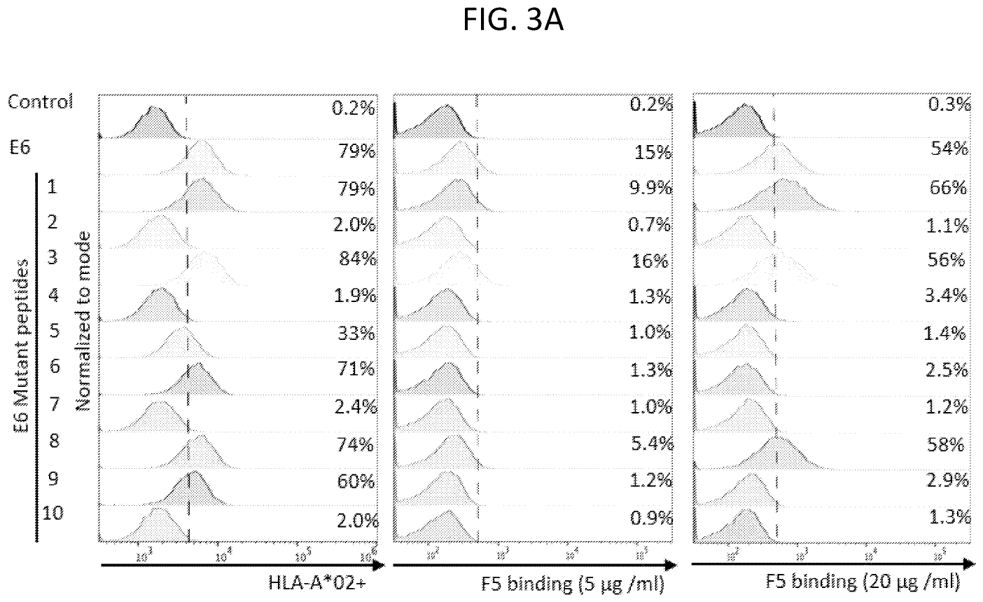

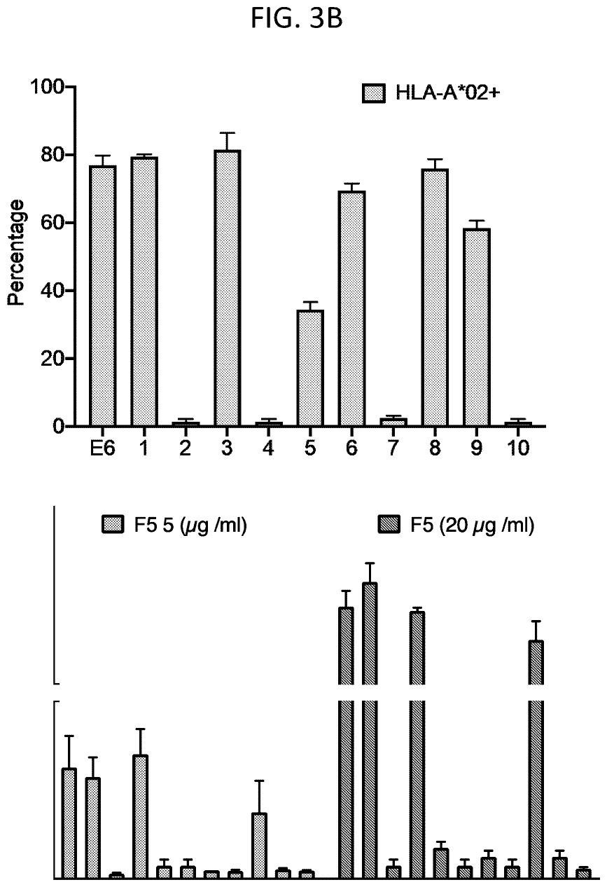

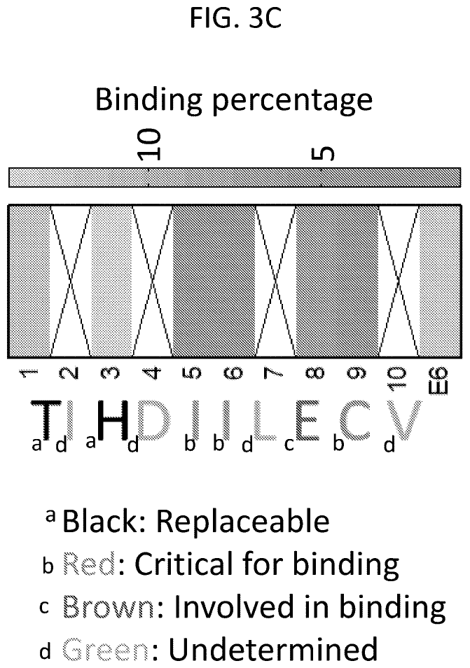

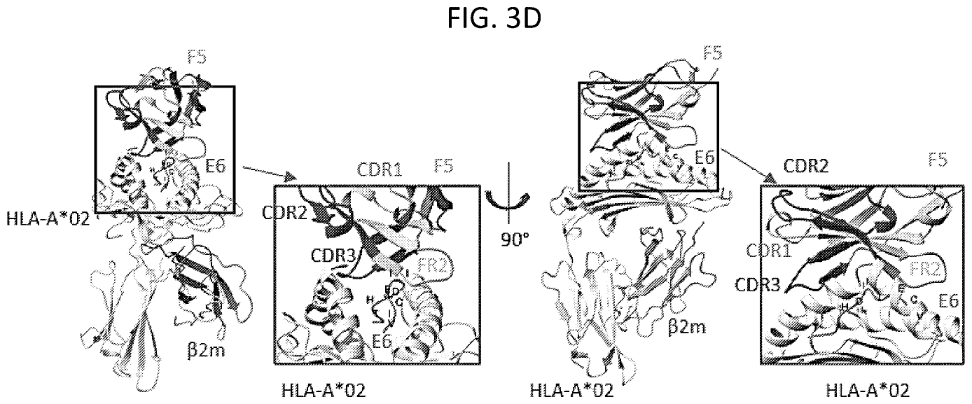

To determine the residues on the peptide that are involved in the binding of F5 and G9 to the complex, an alanine scanning of the E6 peptide was performed. Each amino acid of E6 peptide (TIHDIILECV; SEQ ID NO: 11) was mutated to alanine and synthesized with a high purity (Table 2). The 10 mutated peptides were pulsed on T2 cells and the binding of F5 to the complex was measured by FACS. Mutations at positions 2, 4, 7 and 9 of SEQ ID NO: 11 made the pulsing assay unsuccessful because no stable complex was measured by FACS (FIGS. 3A and 3B), suggesting those 4 sites are important for the complex formation. Therefore, their role in F5 binding could be determined here. Mutations at positions 1 and 3 of SEQ ID NO: 11 did not affect the binding of F5, suggesting that those two residues are replaceable and might not be involved in binding. Mutations at positions 5, 6 and 9 of SEQ ID NO: 11 resulted in complete loss of F5 binding to the complex, indicating that those residues are necessary for binding. Mutation at residue 8 of SEQ ID NO: 11 led to a decreased binding of F5 to the complex, which was more apparent at low F5 concentration (5 μg/ml). It is possible that residue 8 is involved in binding, but plays a less important role than residues 5, 6 and 9 (FIGS. 3A-3C).

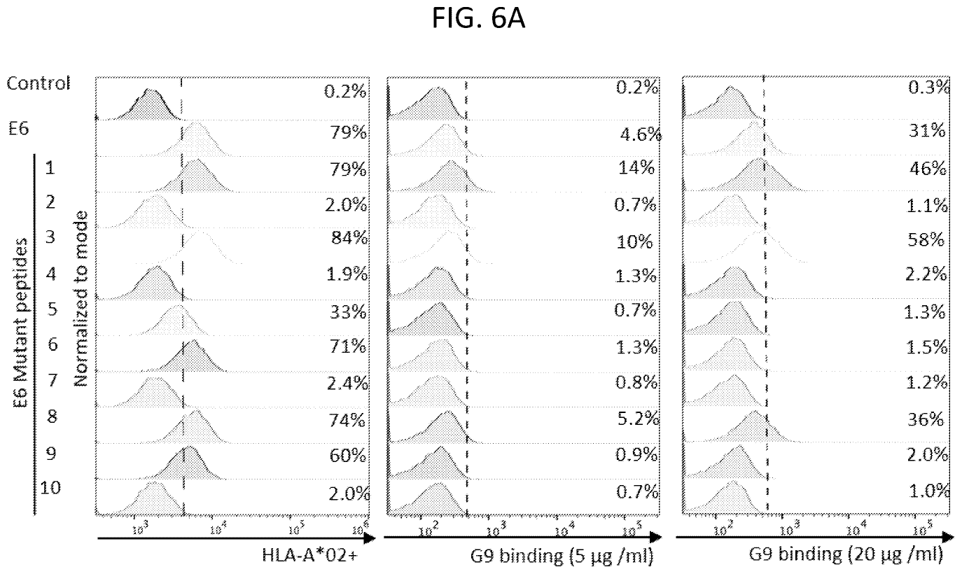

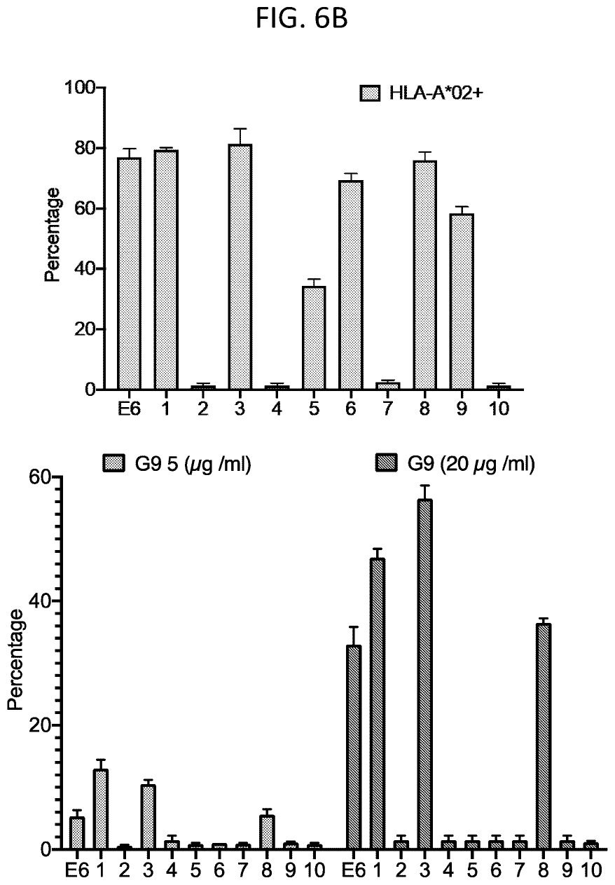

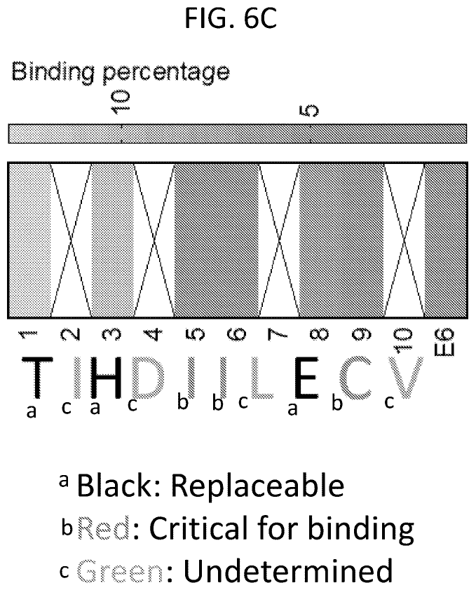

The mutation binding pattern of G9 is similar to that of F5, but it exhibited some differences (FIG. 6). Like F5, G9 did not bind to the complexes with mutations at residues 5, 6 and 9 SEQ ID NO: 11. Additionally, residue 8 of SEQ ID NO: 11 was replaceable for G9 binding together with residues 1 and 3 of SEQ ID NO: 11. In addition, the structures of V_{H}Hs and the complex were predicted by iTASSER (Yang et al., Nat Methods 12(1):7-8, 2015), and the antibody-antigen docking was done with ClusPro (Kozakov et al., Nat Protoc 12(2):255-278, 2017) (FIG. 3D). Predicted models showed that the framework 2 (FR2) portion of F5 might be involved in binding to the peptide C-terminal region. In addition, FR2 and CDR3 of F5 might bind to the HLA region around the peptide binding grove (FIG. 3D). However, G9 prediction did not show similar binding pattern to F5 and instead was predicted to bind HLA molecules only.

TABLE 2
The sequences and purity of E6
and its mutant peptides

| | | SEQ | |
|---|---|---|---|
| | | ID | HPLC |
| Peptide | Sequence* | NO: | purity |
| E6 | TIHDIILECV | 11 | 96.2% |
| 1 | AIHDIILECV | 12 | 97.1% |
| 2 | TAHDIILECV | 13 | 95.9% |
| 3 | TIADIILECV | 14 | 98.0% |
| 4 | TIHAIILECV | 15 | 97.9% |
| 5 | TIHDAILECV | 16 | 96.2% |
| 6 | TIHDIALECV | 17 | 95.8% |
| 7 | TIHDIIAECV | 18 | 96.2% |
| 8 | TIHDIILACV | 19 | 95.1% |
| 9 | TIHDIILEAV | 20 | 98.5% |
| 10 | TIHDIILECA | 21 | 97.7% |

*Alanine substitutions are underlined

### Example 5: Cytotoxicity of F5 and G9 CAR-T Cells In Vitro

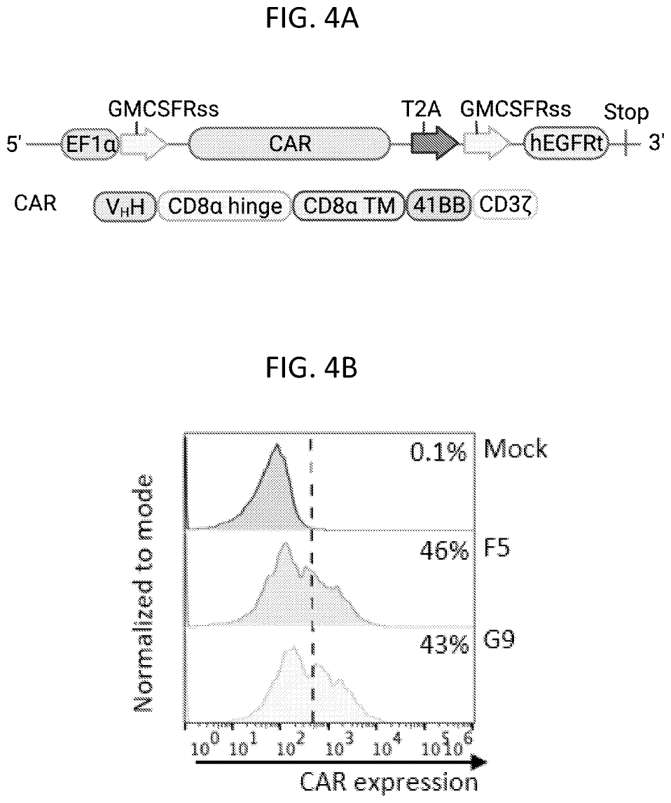

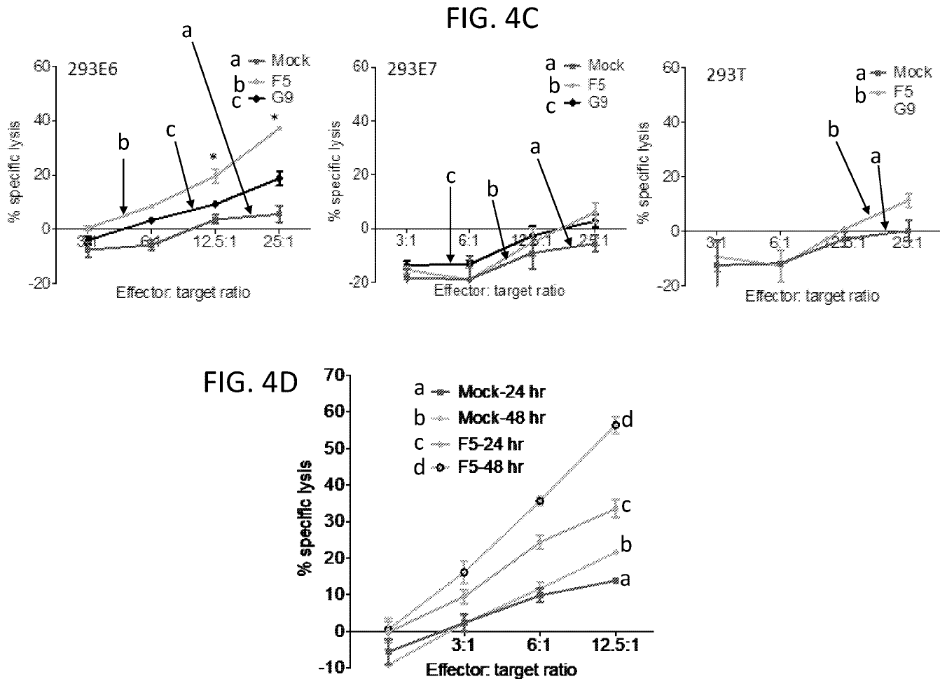

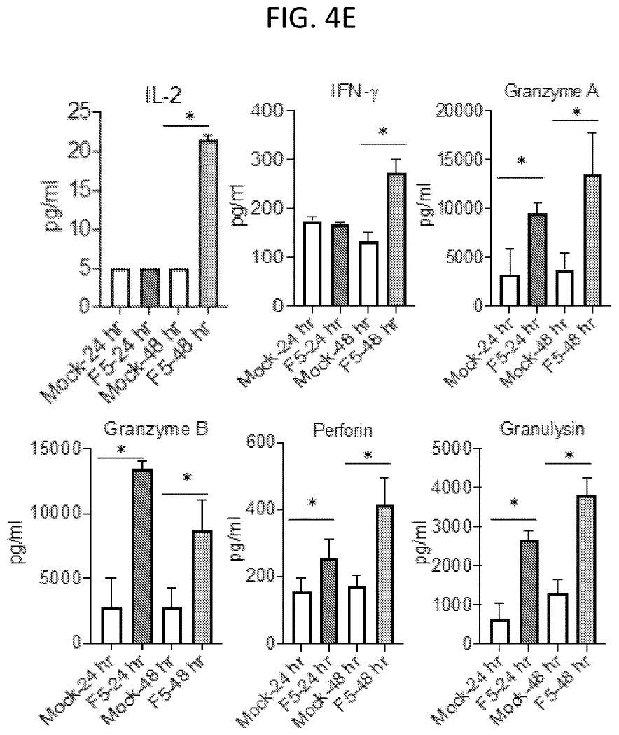

To demonstrate whether F5 and G9 can be used therapeutically for CAR-T treatment, CARs were constructed that contain F5 or G9 as the antigen recognition region, 4-1BB and CD34 signaling domains, and a truncated human EGFR cassette to gauge the transduction efficiency and to inactivate CAR (FIG. 4A). The transduction efficiency of F5 and G9 CAR-T cells was close to 50% (FIG. 4B). Target cells including 293E6, 293E7, 293T and Caski, were transduced with lentiviral GFP/luciferase and used for a luciferase-based cytolytic assay. Both mock T and CAR (F5 and G9) T cells were incubated with target cells for 24 hours. As shown in FIG. 4C, 293E6 cells were specifically lysed by F5 CAR-T cells in a dose-dependent manner compared with mock T cells. The killing ability of G9 was much weaker than F5 and thus was excluded in the following studies.

F5 CAR-T cells were incubated with Caski cells at different effector/target cells (E/T) ratios for an extended time (48 hours) and robust killing was observed with the E/T ratios at 12.5:1 and 6:1 (FIG. 4D). Significantly higher levels of IL-2, IFN-γ, granzymes A and B, perforin and granulysin were released from CAR-T cells when co-cultured with tumor cells for 24 or 48 hours at 12:1 E/T ratios, while minimum cytokine production was observed from mock T cells (FIG. 4E). These results indicated that F5 derived CAR-T cells were able to efficiently lyse Caski tumor cells. Taken together, these results show that F5 CAR-T cells specifically lyse E6-MHC positive human tumor cells.

### Example 6: Inhibition of Caski Xenograft Growth by F5 CAR-T

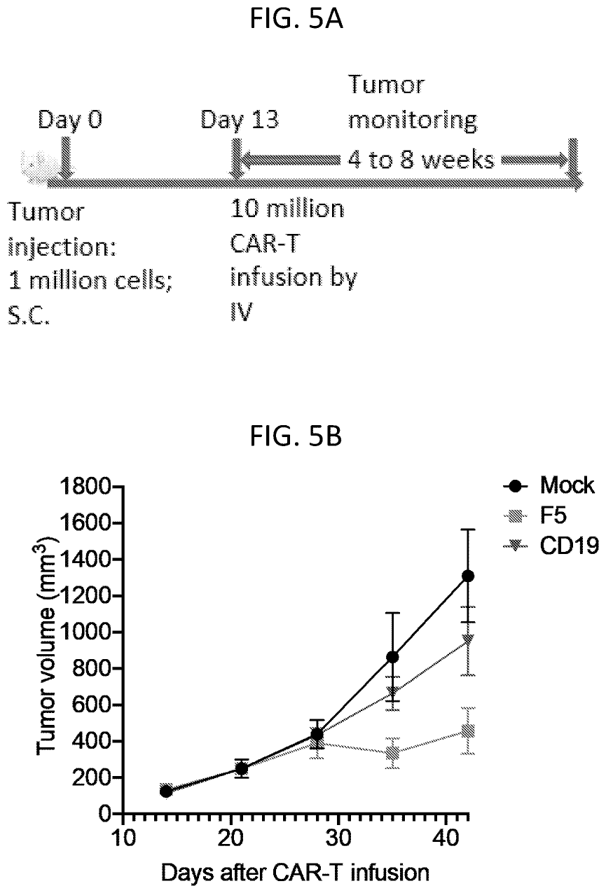

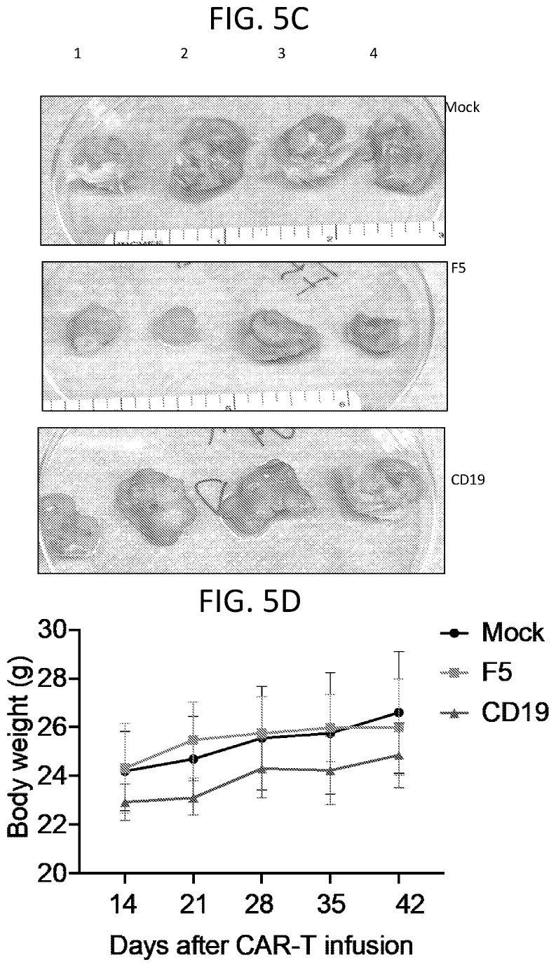

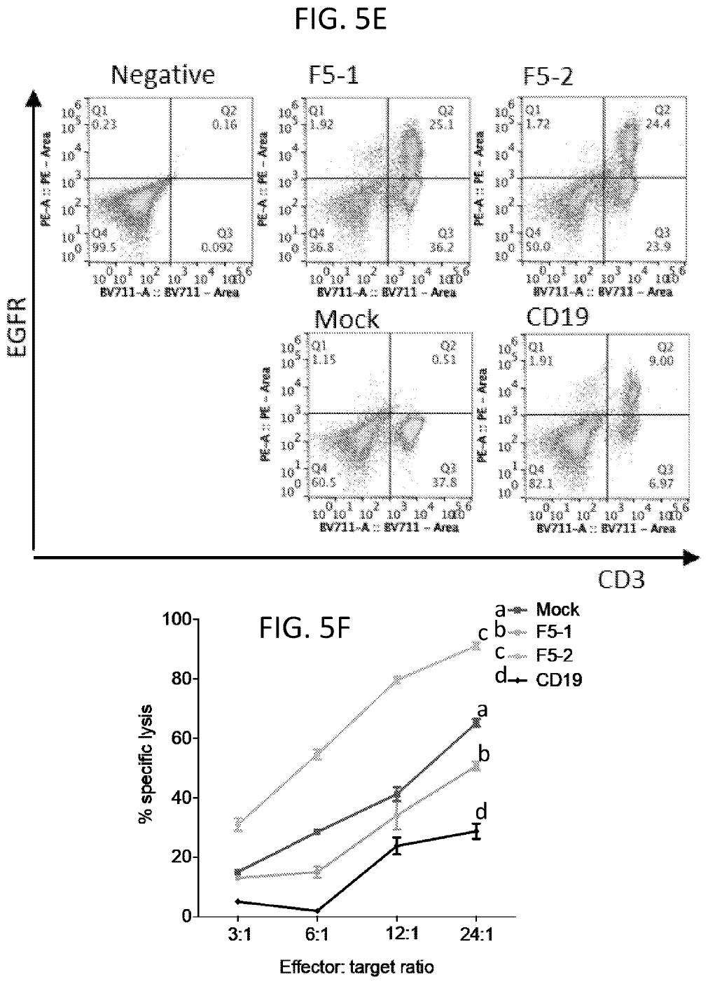

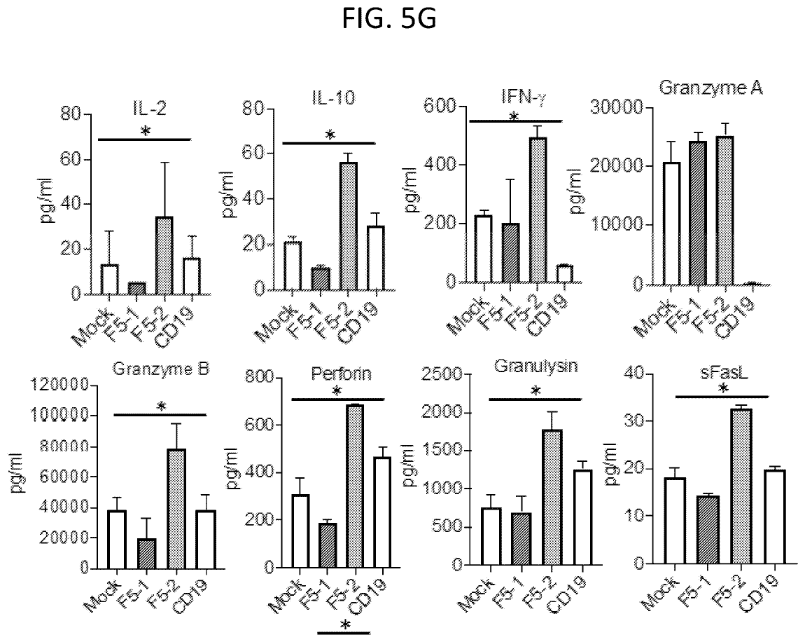

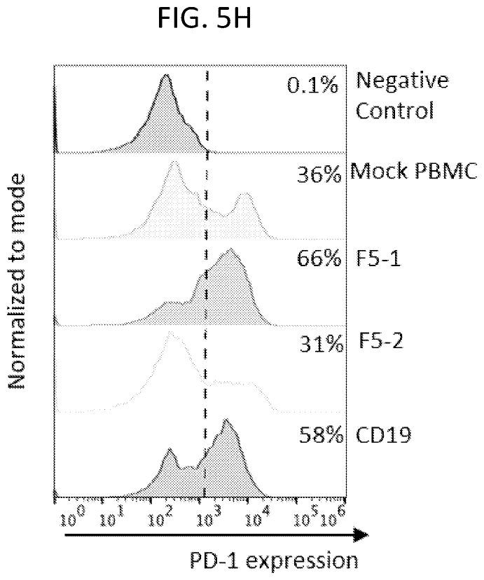

To evaluate the anti-tumor efficacy of F5 CAR-T cells in mice, a HPV16+ cervical cancer xenograft model was established by subcutaneously injecting 1 million Caski luciferase expressing cells into the flank area of the mice. Fourteen days after tumor inoculation, mice were intravenously infused with either F5 CAR-T cells, antigen mismatched CD19 CAR-T cells, or mock cells. The tumor volumes were monitored for seven weeks after CAR-T cell infusion (FIG. 5A). As shown in FIGS. 5B-5D, F5 CAR-T cells reduced tumor burden without a marked loss of body weight compared with the mock and CD19 CAR-T controls. The efficacy of tumor inhibition in the F5 CAR-T treated group was not uniform among the mice, suggesting the heterogenicity of the xenograft tumor and uneven responses of CAR-T treatment. To determine CAR-T persistence, CAR-T cells were recovered from mouse spleen. Ex vivo F5 CAR-T cells recovered from mice had a comparable persistence 6 weeks after infusion (FIG. 5E). The spleen-isolated F5 CAR-T cells from number 2 mouse (F5-2) still showed significant ex vivo cytotoxicity against Caski cells while number 1 mouse (F5-1) did not, compared to control and CD19 cells (FIG. 5F). The cytotoxicity of F5-2 was well correlated with higher release of cytokines, including IL-2, IFN-γ, granzyme B, perforin and granulysin (FIG. 5G) compared with others. In addition, cells from F5-1 showed higher PD-1 expression than F5-2 cells did, which indicated that, unlike F5-2 cells, F5-1 cells had been exhausted and thus could not kill Caski cells efficiently (FIG. 5H).

### Example 7: F5 CAR-T Cells Inhibit Tumor Growth in a Cervical Cancer Xenograft Model

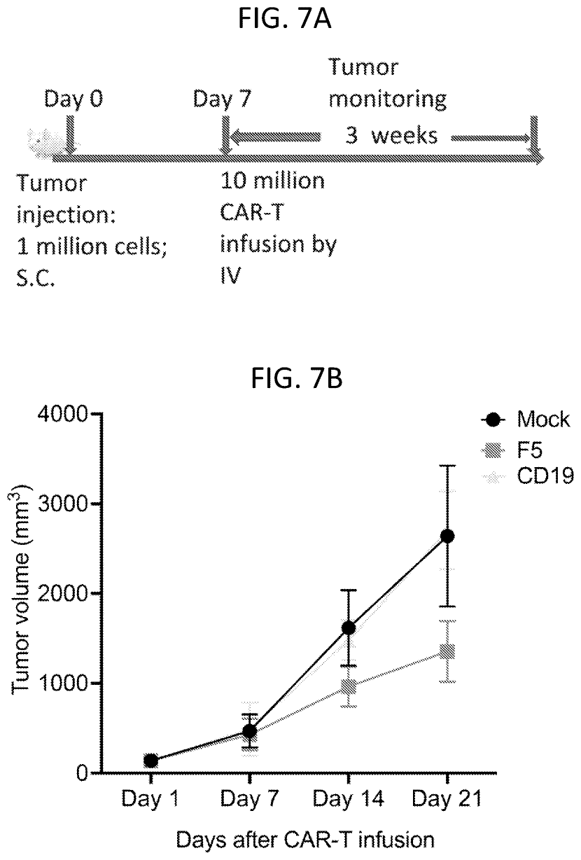

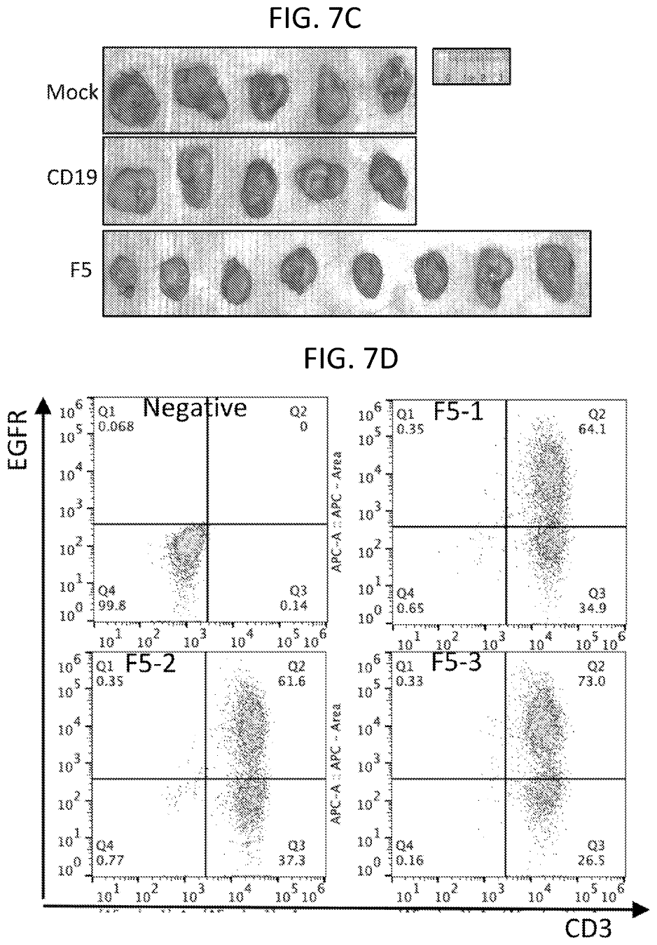

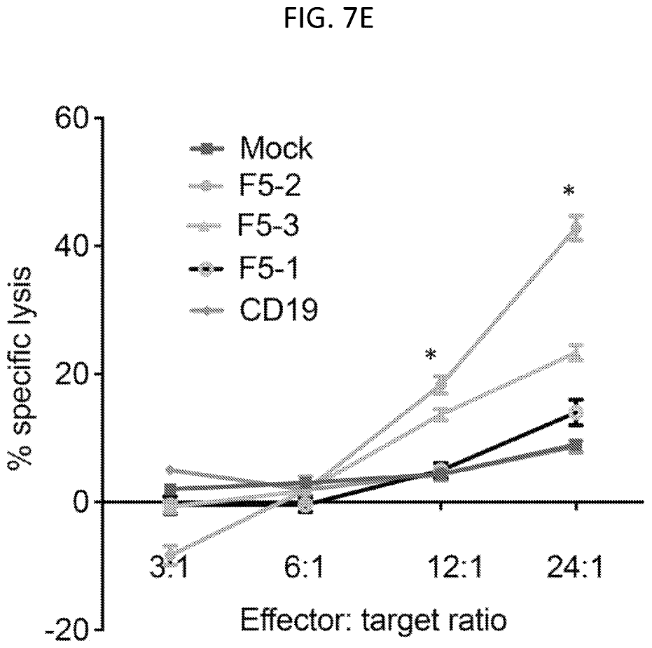

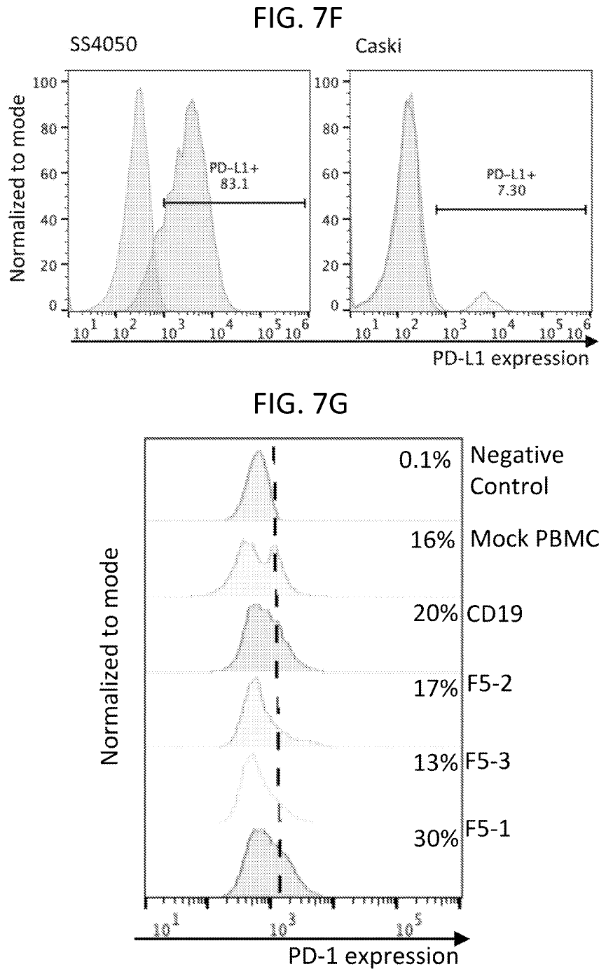

The efficacy of F5 CAR-T was further tested in an additional cervical cancer xenograft model-SS4050. On Day 0, mice were injected subcutaneously with 1 million HPV16-positive cervical tumor cells (SS4050). On Day 7, mice were mock-infused or infused with 10 million F5 CAR-T cells or CD19 CAR-T cells and tumors were monitored for three weeks (FIG. 7A).

Tumor volume was measured by caliper on Days 1, 7, 14 and 21 post CAR T-cell infusion. As shown in FIGS. 7B and 7C, mice treated with F5 CAR-T cells had smaller tumors than mock-treated and CD19 CAR-T cell treated animals.

The spleens of treated mice were harvested on Day 30 and cultured in media containing IL-7, IL-15, and IL-21 to allow for expansion of CAR-T cells. Expression of EGFR and CD3 was measured by flow cytometry after 4 days of culture (FIG. 7D). In addition, the expanded CAR-T cells were cocultured with Caski cells at different E/T ratio for 24 hours and cell viability was measured by luciferase activity (FIG. 7E).

PD-L1 expression in SS4050 and Caski cells was also measured by flow cytometry. As shown in FIG. 7F, SS4050 cells express PD-L1 while Caski cells do not. The isolated spleen cells were also stained with anti-PD-1 antibody by FACS after 4 days of culture (FIG. 7G).

It will be apparent that the precise details of the methods or compositions described may be varied or modified without departing from the spirit of the described aspects of the disclosure. We claim all such modifications and variations that fall within the scope and spirit of the claims below.

1. A polypeptide that specifically binds a human papillomavirus (HPV) E6 or E7 peptide in complex with an MHC class I molecule, wherein the polypeptide comprises the complementarity determining region 1 (CDR1), CDR2 and CDR3 sequences of SEQ ID NO: 1, SEQ ID NO: 2, SEQ ID NO: 3, SEQ ID NO: 4 or SEQ ID NO: 5.

2. The polypeptide of claim 1, wherein the CDR1, CDR2 and CDR3 sequences respectively comprise:
(a) residues 26-33, 51-58 and 97-106 of SEQ ID NO: 1;
(b) residues 31-34, 50-66 and 99-106 of SEQ ID NO: 1;
(c) residues 27-35, 47-60 and 97-107 of SEQ ID NO: 1;
(d) residues 26-33, 51-59 and 97-106 of SEQ ID NO: 2;
(e) residues 31-34, 50-66 and 99-107 of SEQ ID NO: 2;
(f) residues 27-35, 47-60 and 97-107 of SEQ ID NO: 2;
(g) residues 26-33, 51-59 and 96-111 of SEQ ID NO: 3;
(h) residues 33-35, 50-65 and 98-111 of SEQ ID NO: 3;
(i) residues 27-35, 47-59 and 97-112 of SEQ ID NO: 3;
(j) residues 26-33, 51-57 and 96-111 of SEQ ID NO: 4;
(k) residues 33-35, 50-65 and 98-111 of SEQ ID NO: 4;
(l) residues 27-35, 47-59 and 97-112 of SEQ ID NO: 4;
(m) residues 26-33, 51-57 and 96-111 of SEQ ID NO: 5;
(n) residues 33-35, 50-65 and 98-111 of SEQ ID NO: 5; or
(o) residues 27-35, 47-59 and 97-112 of SEQ ID NO: 5.

3-6. (canceled)

7. The polypeptide of claim 1, wherein the amino acid sequence of the polypeptide is at least 90% identical to SEQ ID NO: 1, SEQ ID NO: 2, SEQ ID NO: 3, SEQ ID NO: 4 or SEQ ID NO: 5.

8. The polypeptide of claim 1, wherein the amino acid sequence of the polypeptide comprises or consists of SEQ ID NO: 1, SEQ ID NO: 2, SEQ ID NO: 3, SEQ ID NO: 4 or SEQ ID NO: 5.

9. The polypeptide of claim 1, wherein the polypeptide is a single-domain monoclonal antibody.

10. The polypeptide of claim 9, wherein the single-domain monoclonal antibody is a humanized single-domain monoclonal antibody or a chimeric single-domain monoclonal antibody.

11. (canceled)

12. A composition comprising at least two different polypeptides of claim 1.

13. The composition of claim 12, further comprising a pharmaceutically acceptable carrier.

14. A fusion protein comprising the polypeptide of claim 1 and a heterologous protein.

15-16. (canceled)

17. A chimeric antigen receptor (CAR) comprising the polypeptide of claim 1.

18. The CAR of claim 17, further comprising:
a hinge region;
a transmembrane domain;
an intracellular co-stimulatory domain; and/or
an intracellular signaling domain.

19. The CAR of claim 18, wherein:
the hinge region is an IgG4, CD8 or CD28 hinge region;
the transmembrane domain is a CD8 or CD28 transmembrane domain;
the co-stimulatory domain comprises a 4-1BB signaling moiety; and/or
the signaling domain comprises a CD3ζ signaling domain.

20-22. (canceled)

23. An isolated cell expressing the CAR of claim 17.

24. The isolated cell of claim 23, wherein the cell is an immune cell or an induced pluripotent stem cell (iPSC).

25. (canceled)

26. A soluble T cell receptor (TCR) comprising the polypeptide of claim 1.

27. The soluble TCR of claim 26, further comprising:
an extracellular domain of a TCR alpha chain constant region and an extracellular domain of a TCR beta chain constant region; or
an extracellular domain of a TCR gamma chain constant region and an extracellular domain of a TCR delta chain constant region.

28. An immunoconjugate comprising the polypeptide of claim 1 and an effector molecule.

29. (canceled)

30. An antibody-drug conjugate (ADC) comprising a drug conjugated to the polypeptide of claim 1.

31. A multi-specific antibody comprising the polypeptide of claim 1 and at least one additional monoclonal antibody or antigen-binding fragment thereof.

32-33. (canceled)

34. The multi-specific antibody of claim 31, wherein:
the polypeptide specifically binds an HPV E6 peptide in complex with an MHC class I molecule, and the additional monoclonal antibody or antigen-binding fragment thereof specifically binds an HPV E7 peptide in complex with an MHC class I molecule; or
the polypeptide specifically binds an HPV E7 peptide in complex with an MHC class I molecule, and the additional monoclonal antibody or antigen-binding fragment thereof specifically binds an HPV E6 peptide in complex with an MHC class I molecule.

35. An antibody-nanoparticle conjugate, comprising a nanoparticle conjugated to the polypeptide of claim 1.

36. (canceled)

37. An isolated nucleic acid molecule encoding the polypeptide of claim 1.

38. The isolated nucleic acid molecule of claim 37, comprising the nucleotide sequence of any one of SEQ ID NOs: 6-10, or a degenerate variant thereof.

39. The isolated nucleic acid molecule of claim 37, operably linked to a promoter.

40. A vector comprising the nucleic acid molecule of claim 37.

41. An isolated host cell comprising the vector of claim 40.

42. A composition comprising a pharmaceutically acceptable carrier and the polypeptide of claim 1.

43. A method of treating a human papillomavirus (HPV)-associated cancer or pre-cancerous lesion in a subject, comprising administering to the subject a therapeutically effective amount of the polypeptide of claim 1, thereby treating the HPV-associated cancer or pre-cancerous lesion.

44. The method of claim 43, wherein the HPV-associated cancer a cervical cancer, vaginal cancer, vulvar cancer, penile cancer, anal cancer, or oropharyngeal cancer.

45-46. (canceled)

47. A method of detecting an HPV-infected cell in a sample containing cells, comprising:
contacting the sample with the polypeptide of claim 1; and
detecting binding of the polypeptide to cells in the sample, thereby detecting an HPV-infected cell.

48. A method of diagnosing a subject as having an HPV infection, comprising:
contacting a sample containing cells obtained from the subject with the polypeptide of claim 1; and
detecting binding of the polypeptide to cells in the sample, thereby diagnosing the subject as having an HPV infection.

49. A solid support comprising one or more polypeptides of claim 1.

50. (canceled)


SEQUENCE LISTING (22 sequences)

SEQ ID NO: 1 - protein, 117 aa, synthetic construct
EVQLVESGGGSVQAGGSLRLSCAASGFTFSNNRMGWVRQAPGKGLEWVSDINSSGGVTEYADSVKGRFTISRDNAKNTLYLQLNSLKIEDTAIYYCVYYSYLMRLRPGQGTQVTVSS

SEQ ID NO: 2 - protein, 117 aa, synthetic construct
EVQLVESGGGLVQPGGSLRLSCVASGFTFSNNRMGWVRQAPGKGLEWVSDINSSGGVTEYADSVKGRFTISRDNAKNTLYLQLNSLKIEDTAIYYCVYYSYLMRLRPGQGTQVTVSS

SEQ ID NO: 3 - protein, 122 aa, synthetic construct
AVQLVESGGGLVQPGGSLRLSCAASGFTFSAFAMNWLRQAPGKGLEWVAGISGTPSTYYADSVKGRFTISRDNAKSTLYLQLNSLKTEDTGMYYCSQDRSYFVSVGQIGGTRGQGTQVTVSS

SEQ ID NO: 4 - protein, 122 aa, synthetic construct
QVQLVESGGGSVQAGGSLRLSCASSGFTFSAFAMNWLRQAPGKGLEWVAGISGTPSTYYADSVKGRFTISRDNAKSTLYLQLNSLKTEDTGMYYCSQDRSYFVSVGQIGGTRGQGTQVTVSS

SEQ ID NO: 5 - protein, 122 aa, synthetic construct
EVQLVESGGGLVQPGGSLRLSCAASGFTFSAFAMNWLRQAPGKGLEWVAGISGTPSTYYADSVKGRFTISRDNAKSTLYLQLNSLKTEDTGMYYCSQDRSYFVSVGQIGGTRGQGTQVTVSS

SEQ ID NO: 6 - DNA, 351 bp, synthetic construct
  source 1..351: mol_type = other DNA
gaggtgcagctggtggagtctgggggaggctcggtgcaggctggagggtctctgagactctcctgtgcagcgtctggattcaccttcagtaacaatcgtatgggctgggtccgccaggctccagggaaggggctcgagtgggtctcagatattaatagtagtggtggtgtcactgagtatgcagactccgtgaagggccggttcaccatctccagagacaacgccaagaacacgctgtatctgcaattgaacagcctgaaaattgaggacacggccatctattactgtgtttattatagttatctcatgcgactgcgcccgggccaggggacccaggtcaccgtctcctca

SEQ ID NO: 7 - DNA, 351 bp, synthetic construct
  source 1..351: mol_type = other DNA
gaggtgcagctggtggagtctgggggaggcttggtgcagcccggggggtctctgagactctcctgtgtagcctctggattcaccttcagtaacaatcgtatgggctgggtccgccaggctccagggaaggggctcgagtgggtctcagatattaatagtagtggtggtgtcactgagtatgcagactccgtgaagggccggttcaccatctccagagacaacgccaagaacacgctgtatctgcaattgaacagcctgaaaattgaggacacggccatctattactgtgtttattatagttatctcatgcgactgcgcccgggccaggggacccaggtcaccgtctcctca

SEQ ID NO: 8 - DNA, 366 bp, synthetic construct
  source 1..366: mol_type = other DNA
gcggtgcagctggtggagtctgggggaggcttggtgcagcccggggggtctctgagactctcctgtgcagcctctggattcaccttcagtgcctttgccatgaactggctccgccaggctccagggaaggggctcgagtgggtcgcaggtattagtggtactcccagtacatactatgcagactccgtgaagggccgattcaccatctccagagacaacgccaagagtacgctgtatctgcaattgaacagcctgaaaaccgaagacacgggcatgtattactgttcgcaagatcgttcttattttgtctcggttggccaaatcggcgggacgaggggccaggggacccaggtcaccgtctcctca

SEQ ID NO: 9 - DNA, 366 bp, synthetic construct
  source 1..366: mol_type = other DNA
caggtgcagctggtggagtctgggggaggctcggtgcaggctggagggtccctgagactctcctgtgcatcctctggattcaccttcagtgcctttgccatgaactggctccgccaggctccagggaaggggctcgagtgggtcgcaggtattagtggtactcccagtacatactatgcagactccgtgaagggccgattcaccatctccagagacaacgccaagagtacgctgtatctgcaattgaacagcctgaaaaccgaagacacgggcatgtattactgttcgcaagatcgttcttattttgtctcggttggccaaatcggcgggacgaggggccaggggacccaggtcaccgtctcctca

SEQ ID NO: 10 - DNA, 366 bp, synthetic construct
  source 1..366: mol_type = other DNA
gaggtgcagctggtggagtctgggggaggcttggtgcagcccggggggtctctgagactctcctgtgcagcctctggattcaccttcagtgcctttgccatgaactggctccgccaggctccagggaaggggctcgagtgggtcgcaggtattagtggtactcccagtacatactatgcagactccgtgaagggccgattcaccatctccagagacaacgccaagagtacgctgtatctgcaattgaacagcctgaaaaccgaagacacgggcatgtattactgttcgcaagatcgttcttattttgtctcggttggccaaatcggcgggacgaggggccaggggacccaggtcaccgtctcctca

SEQ ID NO: 11 - protein, 10 aa, synthetic construct
TIHDIILECV

SEQ ID NO: 12 - protein, 10 aa, synthetic construct
AIHDIILECV

SEQ ID NO: 13 - protein, 10 aa, synthetic construct
TAHDIILECV

SEQ ID NO: 14 - protein, 10 aa, synthetic construct
TIADIILECV

SEQ ID NO: 15 - protein, 10 aa, synthetic construct
TIHAIILECV

SEQ ID NO: 16 - protein, 10 aa, synthetic construct
TIHDAILECV

SEQ ID NO: 17 - protein, 10 aa, synthetic construct
TIHDIALECV

SEQ ID NO: 18 - protein, 10 aa, synthetic construct
TIHDIIAECV

SEQ ID NO: 19 - protein, 10 aa, synthetic construct
TIHDIILACV

SEQ ID NO: 20 - protein, 10 aa, synthetic construct
TIHDIILEAV

SEQ ID NO: 21 - protein, 10 aa, synthetic construct
TIHDIILECA

SEQ ID NO: 22 - protein, 9 aa, synthetic construct
YMLDLQPET